(19) United States
(12) Patent Application Publication
(10) Pub. No.: US 2025/0186023 A1
(43) Pub. Date: Jun. 12, 2025

(54) ELECTRONIC DEVICE, MEDICAL INSTRUMENT, AND ULTRASONIC DIAGNOSTIC APPARATUS

(71) Applicant: CANON KABUSHIKI KAISHA, Tokyo (JP)

(72) Inventors: YUKI NIWAMAE, Kanagawa (JP); SEISHIRO TAKANO, Kanagawa (JP); TAKAHIRO YAMAMOTO, Kanagawa (JP); NOBUO OSHIMOTO, Tokyo (JP); SAKIKO TANIMOTO, Shizuoka (JP); SATOSHI SHINATA, Kanagawa (JP); TETSUYA SEKINE, Kanagawa (JP); SHOSAKU KAWASHIMA, Kanagawa (JP); MEGUMI MATSUDA, Kanagawa (JP)

(21) Appl. No.: 19/060,134

(22) Filed: Feb. 21, 2025

Related U.S. Application Data
(63) Continuation of application No. PCT/JP2023/027057, filed on Jul. 24, 2023.

(30) Foreign Application Priority Data
Aug. 24, 2022 (JP) 2022-133580
Aug. 24, 2022 (JP) 2022-133655

(51) Int. Cl. A61B 8/00

(52) U.S. Cl. CPC A61B 8/4411; A61B 8/54

(57) ABSTRACT

An electronic device according to the present disclosure includes a base material, a processor, and a memory storing a program which causes the image processing apparatus to: execute detection processing of detecting an operation performed on the base material by an operator, acquisition processing of acquiring, on the basis of an output from the detection processing according to the detection of the operation, position information related to the operation on the base material, and control processing of controlling a behavior of the electronic device on the basis of the position information acquired by the acquisition processing, wherein the base material is provided with a region where in the control processing, the operation by the operator is not received.

### CROSS-REFERENCE TO RELATED APPLICATIONS

This application is a Continuation of International Patent Application No. PCT/JP2023/027057, filed on Jul. 24, 2023, which claims the benefits of Japanese Patent Application No. 2022-133580, filed on Aug. 24, 2022 and Japanese Patent Application No. 2022-133655, filed on Aug. 24, 2022, all of which are hereby incorporated by reference herein in their entirety.

### BACKGROUND OF THE INVENTION

### Field of the Invention

The present invention relates to an electronic device, a medical instrument, and an ultrasonic diagnostic apparatus which control a behavior of various equipment.

### Background Art

As a conventional technology, a control interface is disclosed, which specifies a position of a pressure, placed by a finger of an operator, on an outer surface made of a rigid plate (PTL 1). On the basis of measurement values supplied by at least three force sensors configured to measure a force in a direction substantially perpendicular to a plane of the rigid plate, the position of the finger pressure can be calculated. In addition, with a display screen placed to face the rigid plate and the transparent rigid plate, the operator can push the rigid plate with his finger to select and operate a plurality of functions displayed on the screen.

In the control interface in PTL 1, the rigid plate serving as the outer surface is planar, and therefore the operator cannot identify function regions without visually recognizing the display screen, hence operation thereof is difficult.

Therefore, the technology according to the present disclosure aims at providing an electronic device, a medical instrument, and an ultrasonic diagnostic apparatus each having an improved operability.

### CITATION LIST

### Patent Literature

PTL 1 Japanese Patent Application Laid-open No. 2015-537321

### SUMMARY OF THE INVENTION

According to some embodiments, an electronic device includes a base material; a processor; and a memory storing a program which, when executed by the processor, causes the image processing apparatus to execute detection processing of detecting an operation performed on the base material by an operator; execute acquisition processing of acquiring, on the basis of an output from the detection processing according to the detection of the operation, position information related to the operation on the base material; and execute control processing of controlling a behavior of the electronic device on the basis of the position information acquired by the acquisition processing, wherein the base material is provided with a region where in the control processing, the operation by the operator is not received.

According to some embodiments, a medical instrument includes a base material; a processor; and a memory storing a program which, when executed by the processor, causes the medical instrument to execute detection processing of detecting an operation performed on the base material by an operator; execute acquisition processing of acquiring, on the basis of an output from the detection processing according to the detection of the operation, position information related to the operation on the base material; and execute control processing of controlling a behavior of the medical instrument on the basis of the position information acquired by the acquisition processing, wherein the base material is provided with a region where in the control processing, the operation by the operator is not received.

According to some embodiments, an ultrasonic diagnostic apparatus includes a base material; a processor; and a memory storing a program which, when executed by the processor, causes the ultrasonic diagnostic apparatus to execute detection processing of detecting an operation performed on the base material by an operator; execute acquisition processing of acquiring, on the basis of an output from the detection processing according to the detection of the operation, position information related to the operation on the base material; and execute control processing of controlling a behavior of the ultrasonic diagnostic apparatus on the basis of the position information acquired by the acquisition processing, wherein the base material is provided with a region where in the control processing, the operation by the operator is not received.

Further features of the present invention will become apparent from the following description of exemplary embodiments with reference to the attached drawings.

### BRIEF DESCRIPTION OF THE DRAWINGS

FIG. 1 illustrates a diagram illustrating a configuration of an ultrasonic diagnostic apparatus according to a first embodiment.

FIG. 2A illustrate a perspective view and FIG. 2B illustrates a top view each view of which illustrates an outer appearance of the ultrasonic diagnostic apparatus according to the first embodiment.

FIGS. 3A and 3B illustrates illustrative views illustrating coordinates of a sensor and coordinates of a point of action when the apparatus illustrated in FIG. 2 is used.

FIG. 4 illustrates a diagram illustrating an operation map of an acquisition unit according to the first embodiment.

FIG. 5 illustrates a flow chart of operation processing by the acquisition unit according to the first embodiment.

FIG. 6 illustrates an enlarged perspective view of a base material having an ellipsoidal depressed-type stereoscopic portion according to the first embodiment.

FIGS. 7A and 7B illustrate diagrams illustrating an example of a slide switch according to the first embodiment.

FIGS. 8A and 8B illustrate enlarged perspective views of the base material according to the first embodiment.

FIGS. 9A to 9D illustrate diagrams illustrating an example of a slide bar according to the first embodiment.

FIG. 10 illustrates an enlarged perspective view of the base material having a plurality of cone-shaped protruded-type stereoscopic portions according to the first embodiment.

FIG. 11 illustrates an enlarged perspective view of the base material according to the first embodiment.

FIG. 12 illustrates a diagram illustrating an operation region of a dial trackball according to the first embodiment.

FIGS. 13A to 13C illustrate schematic diagrams of a three-dimensional stereoscopic image displayed on a display unit according to the first embodiment.

FIG. 14 illustrates a perspective view of a hemispherical depressed-type stereoscopic portion according to a modification.

FIG. 15 illustrates a perspective view of a rectangular protruded-type stereoscopic portion according to a modification.

FIG. 16 illustrates a perspective view of cross-key-shaped protruded- and depressed-type stereoscopic portions according to a modification.

FIG. 17 illustrates a perspective view of a depressed-type stereoscopic portion and a protruded-type stereoscopic portion according to a modification.

FIG. 18 illustrates a diagram illustrating a configuration of an ultrasonic diagnostic apparatus according to a second embodiment.

FIG. 19 illustrates a top view illustrating an example of an outer appearance of a base material according to the second embodiment.

FIG. 20 illustrates a perspective view illustrating a portion of an outer appearance of the ultrasonic diagnostic apparatus according to the second embodiment.

FIG. 21 illustrates a flow chart of initial setting when the base material is to be replaced according to the second embodiment.

FIG. 22 illustrates a diagram illustrating a configuration of an ultrasonic diagnostic apparatus according to a third embodiment.

FIG. 23 illustrates a perspective view illustrating an example of an outer appearance of the ultrasonic diagnostic apparatus according to the third embodiment.

### DESCRIPTION OF THE EMBODIMENTS

Referring to the drawings, a description will be given of embodiments of the present disclosure. Note that the present disclosure is not limited to the following embodiments, and can appropriately be changed within the scope not departing from the gist thereof. In addition, in the drawings described below, components having the same functions may be denoted by the same reference signs, and a description thereof may be omitted or simplified.

### First Embodiment

Hereinbelow, a description will be given of an ultrasonic diagnostic apparatus including an electronic device according to the first embodiment.

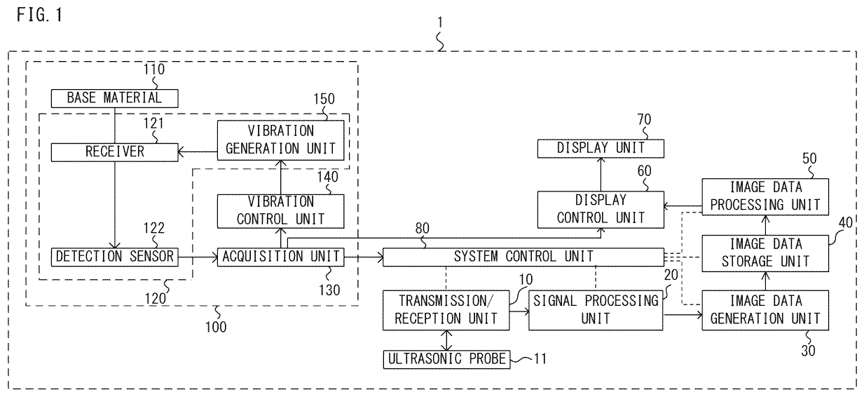

(Configuration) FIG. 1 is a diagram illustrating a configuration of the ultrasonic diagnostic apparatus according to the present embodiment. An ultrasonic diagnostic apparatus 1 is an apparatus that uses a reflected wave obtained by projecting an ultrasonic wave onto an object not shown to generate an ultrasonic image.

The ultrasonic diagnostic apparatus 1 includes a transmission/reception unit 10, a signal processing unit 20, an image data generation unit 30, an image data storage unit 40, an image data processing unit 50, a display control unit 60, a display unit 70, and a system control unit 80, and is also an example of a medical instrument. The medical instrument may also be an X-ray device, a CT device, an MRI device, or the like.

An ultrasonic probe 11 of the ultrasonic diagnostic apparatus 1 uses an electric signal as a timing signal and transmits an ultrasonic wave from a piezoelectric vibrator embedded therein to the object, while receiving the reflected wave and converting the received reflected wave to an electric signal (reflected wave signal). The ultrasonic probe 11 is a medical tool for performing at least one of observation, examination, and treatment on the object.

The transmission/reception unit 10 controls transmission and reception of the ultrasonic wave, which is performed by the ultrasonic probe 11. The transmission/reception unit 10 has a transmission delay circuit and the like, and supplies a drive signal to the ultrasonic probe 11. The transmission/reception unit 10 repeatedly generates a rate pulse at a predetermined repetition frequency (PRF). The transmission delay circuit of the transmission/reception unit 10 focuses the ultrasonic wave generated from the ultrasonic probe 11, and gives a delay time for determining a transmission directivity to the rate pulse generated from the transmission unit. The transmission delay circuit changes the delay time to be given to the rate pulse to be able to control a direction of transmission of the ultrasonic wave transmitted from the vibrator.

The transmission/reception unit 10 also has an amplifier, an A/D conversion unit, a reception delay circuit, an addition unit, and the like. The ultrasonic probe 11 performs various processing on the reflected wave signal received thereby to generate an ultrasonic signal. The amplifier amplifies the reflected wave signal for each channel to perform gain correction processing. The A/D conversion unit performs A/D conversion of the reflected wave signal with a corrected gain. The reception delay circuit gives the delay time to digital data in order to determine a reception directivity. The addition unit performs addition processing on the reflected wave signal to which the delay time has been given by the reception delay circuit. By the addition processing by the addition unit, a reflected component from a direction according to the reception directivity of the reflected wave signal is enhanced.

When performing two-dimensional scanning on the object, the transmission/reception unit 10 causes the ultrasonic probe 11 to transmit a two-dimensional ultrasonic wave. Then, the transmission/reception unit 10 generates a two-dimensional ultrasonic signal from the two-dimensional reflected wave signal received by the ultrasonic probe 11. When performing three-dimensional scanning on the object, the transmission/reception unit 10 also causes the ultrasonic probe 11 to transmit a three-dimensional ultrasonic wave. Then, the transmission/reception unit 10 generates a three-dimensional ultrasonic signal from the three-dimensional reflected wave signal received by the ultrasonic probe 11.

The signal processing unit 20 performs various signal processing on the ultrasonic signal output from the transmission/reception unit 10. Specifically, the signal processing unit 20 performs signal processing such as wave detection processing or logarithmic compression on the ultrasonic signal. The signal processing unit 20 images amplitude information of the ultrasonic signal to generate signal processing data (raster data). The signal processing unit 20 performs bandpass filter processing on the ultrasonic signal output from the transmission/reception unit 10, and then detects an envelope of an output signal. Then, the signal processing unit 20 performs compression processing on the detected data by logarithmic conversion. The signal processing unit 20 outputs the signal processing data after the signal processing to the image data generation unit 30.

The image data generation unit 30 uses signal processing data subjected to the signal processing performed by the signal processing unit 20 to generate an ultrasonic image. The image data generation unit 30 has a digital scan converter and converts the signal processing data to data represented by orthogonal coordinates. The image data generation unit 30 performs herein orthogonal conversion of the signal processing data (raster data) to a coordinate system (X, Y) of image data for display. Then, the image data generation unit 30 generates the ultrasonic image (B-mode image data) in which a signal intensity is expressed by luminousness of brightness. Thus, in the image data generation unit 30, the ultrasonic image is generated. As an ultrasonic image generation algorithm, not only phasing addition processing, but also any algorithm is used appropriately to allow image reconstruction to be performed.

The image data generation unit 30 can generate blood flow image data according to a color Doppler method referred to as a color flow mapping method (CFM). In the color Doppler method, an ultrasonic wave is transmitted a plurality of times in the same direction and, by performing frequency analysis based on a Doppler effect, blood flow movement information can be extracted from the received reflected wave signal. The display control unit 60 uses the color Doppler method to generate blood flow information such as an average speed, dispersion, and power as the blood flow image data. Note that the image data generation unit 30 may also generate the blood flow image data according to a power Doppler method.

The image data storage unit 40 receives the image data from the image data generation unit 30 and stores the image data, while the image data processing unit 50 generates a cross-sectional image and a three-dimensional stereoscopic image from the image data stored in the image data storage unit 40.

The display control unit 60 displays the ultrasonic image and a region of interest (ROI) thereof on the display unit 70. In addition, the display control unit 60 receives a signal output from an acquisition unit of the electronic device in response to an operation by an operator, and performs various display processing such as switching of a zoom magnification of the ultrasonic image and switching between an orthogonal 3-section image and a three-dimensional stereoscopic image. The display control unit 60 has a GUI (Graphical User Interface) for the operator to input an operation instruction for each function by using an electronic device 100. Thus, the display control unit 60 changes the zoom magnification of the ultrasonic image on a screen of the display unit 70 in conjunction with, e.g., an operation of the electronic device 100. Details of the GUI of the display control unit 60 with respect to each operation device of the electronic device 100 will be described later.

The system control unit 80 controls various processing to be performed by the transmission/reception unit 10, the signal processing unit 20, the image data generation unit 30, the image data storage unit 40, and the image data processing unit 50 on the basis of an operation signal from an acquisition unit 130 of the electronic device 100.

The electronic device 100 in the present embodiment is attached to the ultrasonic diagnostic apparatus 1. The electronic device 100 includes a base material 110, a detection unit 120, the acquisition unit 130, a vibration control unit 140, and a vibration generation unit 150.

The base material 110 has an outer surface serving as an operation panel of the ultrasonic diagnostic apparatus 1, which is formed with an operation unit formed of at least one of a plurality of protruded portions and depressed portions serving as an operation device such as a switch, a dial trackball, or a slider, as will be described later. The operator touches the operation unit of the base material 110 with his finger to operate the ultrasonic diagnostic apparatus 1. The base material 110 is attached to a receiver 121 of the detection unit 120.

The detection unit 120 is configured to include the receiver 121, a detection sensor 122 that detects at least one of a force acting when the operator touches the base material 110 and a moment, and the vibration generation unit 150, and detects an operation by the operator. The receiver 121 is attached to the detection sensor 122 and configured as an attachment to the base material 110. By way of example, the detection sensor 122 is a six-axis force sensor using a strain gauge. Note that, as the detection sensor 122, not only the six-axis force sensor that can detect the point of action, a position of the force at the point of action, and the moment with one detection sensor, but also a one-axis or three-axis force sensor, a piezoelectric sensor, or a touch sensor (surface pressure sensor) element may also be used.

The acquisition unit 130 acquires a point of action of a finger of the operator on the basis of data resulting from the detection by the detection unit 120. Accordingly, the acquisition unit 130 has a function of acquiring position information related to an operation on the base material on the basis of an output from the detection unit according to the detection of the operation. The acquisition unit 130 uses an operation map 160 in which each operation region is determined according to the protruded portion and/or the depressed portion serving as the operation unit on the base material 110 to determine that there is an operation when the calculated point of action is inside the operation region of the operation map 160. The operation map 160 is function information related to a function performed by at least one operation of the protruded portion and the depressed portion. To each operation region of the operation map 160, a predetermined function for operating the ultrasonic diagnostic apparatus 1 is allocated.

In addition, the acquisition unit 130 is connected to the display control unit 60 and the system control unit 80 of the ultrasonic diagnostic apparatus 1. When determining that there is an operation, the acquisition unit 130 acquires operation information of a predetermined function on the basis of the position of the force and the moment from the detection sensor 122, and outputs the acquired operation information as an operation signal to the display control unit 60 and the system control unit 80. Thus, the acquisition unit 130 acquires information on at least one operation of the protruded portion and the depressed portion by the operator on the basis of at least one of the force and moment each detected by the detection unit. Furthermore, the acquisition unit 130 functions also as a control unit that controls a behavior of the electronic device on the basis of the position information and the function information. The acquisition unit 130 outputs a vibration signal corresponding to the operation information of the predetermined function to the vibration control unit 140.

To notify the operator that the operation unit is being operated, the vibration control unit 140 controls vibration of the vibration generation unit 150 on the basis of the signal from the acquisition unit 130. As the vibration generation unit 150, a vibration actuator such as, e.g., a piezoelectric element or a voice coil motor is used, and the vibration generation unit 150 is attached to the receiver 121 of the detection unit 120. Under the control of the vibration control unit 140, the vibration generation unit 150 vibrates the receiver 121 and gives a force feedback, which is a vibration feedback, to the operator touching the base material 110. This allows the operator to recognize that the operation is performed via the operation unit on the base material 110.

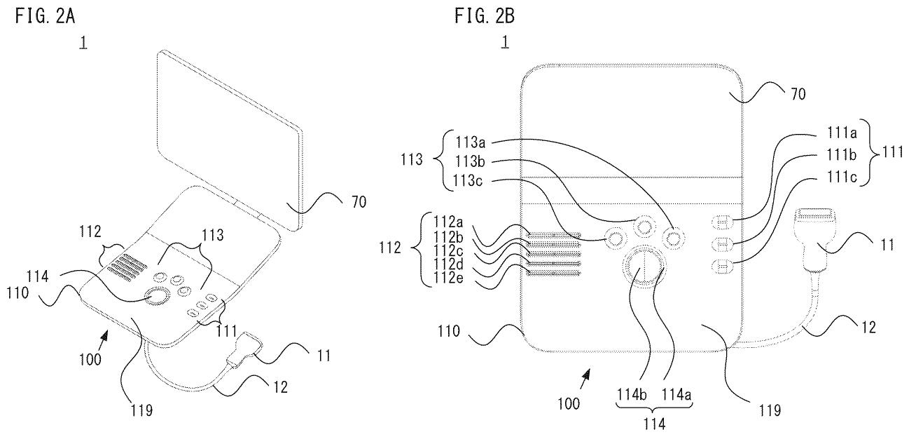

(Outer Appearance) FIG. 2A is a perspective view illustrating an outer appearance of each of the electronic device 100 serving as the operation panel of the ultrasonic diagnostic apparatus 1, the display unit 70, the ultrasonic probe 11, and a cable 12, while FIG. 2B is a top view of the operation panel of the ultrasonic diagnostic apparatus 1. Over the outer surface of the ultrasonic diagnostic apparatus 1, the base material 110 is provided, and the display unit 70 is provided adjacent to the base material 110. Note that the present embodiment shows an example in which the display unit 70 is configured to be integral with the base material 110, but a configuration of the present invention is not limited thereto. For example, it may also be possible to use a configuration in which the display unit 70 is formed of a thin liquid crystal display or the like, and a casing of the ultrasonic diagnostic apparatus 1 and the display unit 70 are connected together with a movable arm to allow an orientation of the display unit 70 to be changed optionally.

The ultrasonic probe 11 is a medical instrument to be connected to the casing of the ultrasonic diagnostic apparatus 1 via the cable 12. The cable 12 is formed of a soft raw material such as, e.g., rubber, silicon, or polyvinyl chloride, and has an electric cable that transmits and receives an electronic signal embedded therein. A drive signal transmitted by the transmission/reception unit 10 is transmitted to the ultrasonic probe 11 via the cable 12, while an echo signal received by the ultrasonic probe 11 is transmitted to the transmission/reception unit 10 via the cable 12. Since the cable 12 is formed of a bendable soft raw material, the operator can bend the cable 12 and move the ultrasonic probe 11 to an intended examination region.

(Electronic Device) The electronic device 100 is disposed as the operation panel of the ultrasonic diagnostic apparatus 1. Over an outer surface of the electronic device 100, the base material 110 is provided, and a surface of the base material 110 opposite to the outer surface is attached to the receiver 121 of the detection unit 120. The detection unit 120 is configured to include the receiver 121, the detection sensor 122, and the vibration generation unit 150 each not shown.

As illustrated in FIG. 2B, the base material 110 has a slide switch unit 111 configured to include ellipsoidal depressed portions 111a, 111b, and 111c and a slider unit 112 configured to include linear protruded portions 112a, 112b, 112c, 112d, and 112e. The base material 110 further has a dial unit 113 configured to include cone-shaped protruded portions 113a, 113b, and 113c. The base material 110 has a dial trackball unit 114 obtained by combining together a circular depressed portion 114a serving as a groove in a planar portion and a protruded portion 114b which is hemispherically protruded inside the depressed portion 114a.

The base material 110 is formed of a material which is not easily deformed by an operation by the operator, e.g., a resin or glass. The base material 110 has the outer surface formed as the integral operation panel. Accordingly, even when a disinfectant such as alcohol is sprayed onto the base material 110, a liquid does not reach the detection sensor 122 of the detection unit 120 and the vibration generation unit 150, which does not result in a failure due to a short circuit of the electronic device 100. In other words, it is possible to spray a liquid such as the disinfectant onto the base material 110 serving as the operation panel to clean the base material 110 and keep the electronic device 100 clean.

In addition, of the base material 110, a planar portion 119 other than the operation units including the slide switch unit 111, the slider unit 112, and the dial unit 113 corresponds to a region where the acquisition unit 130 does not receive an operation even when the operator touches the region. Accordingly, when the operator unintentionally touches the planar portion 119, the detection sensor 122 described below detects a position touched by the operator and a force, but the acquisition unit 130 does not determine this to be an operation. As a result, even when the operator touches the planar portion 119, the electronic device 100 performs no function, and therefore it is possible to prevent an erroneous behavior resulting from unintentional touching of the planar portion 119 by the operator. In addition, when the operator touches the planar portion 119, under the control of the vibration control unit 140, the vibration generation unit 150 does not vibrate the receiver 121, i.e., the base material 110 (planar portion 119). This allows the operator to recognize that he has not operated the electronic device by touching the planar portion 119.

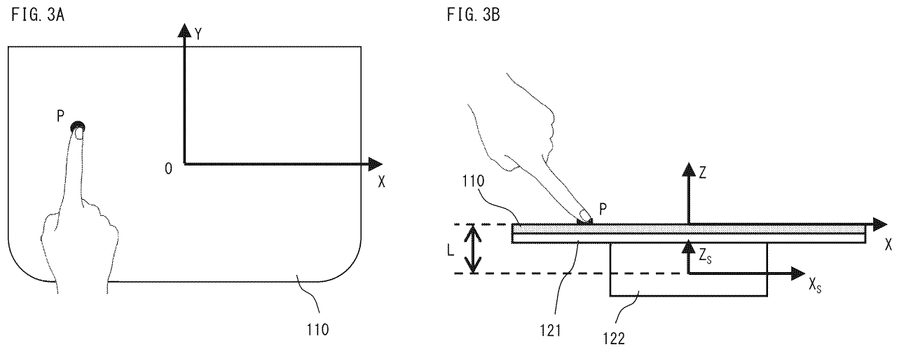

FIG. 3 illustrates illustrative views illustrating coordinates of the detection sensor 122 and coordinates of the point of action when the electronic device 100 illustrated in FIGS. 2A and 2B is used. Note that, in FIG. 3, for illustration purpose, depiction of the protruded portions and the depressed portions of the operation units of the base material 110 is omitted.

The six-axis force sensor as an example of the detection sensor 122 measures at least one of the force and the moment when the operator touches the base material 110 with his finger. The acquisition unit 130 calculates, from the force and/or moment measured by the detection sensor 122, coordinates P (Px, Py, Pz) of the point of action touched by the finger.

As illustrated in FIGS. 3A and 3B, it is assumed that forces of the six-axis force sensor in Xs-, Ys-, and Zs-axis directions are Fx, Fy, and Fz, and moments around the individual axes are Mx, My, and Mz. The coordinates P (Px, Py, Pz) of the point of action of a finger force are calculated according to Expressions (1) to (3) below. It is to be noted herein that L is a distance from an origin of the six-axis force sensor to the surface of the base material. Note that the forces Fx, Fy, and Fz exerted in axial directions are vectors indicating a magnitude and a direction of a touch force.

$$\begin{matrix}[ Math . 1 ] & \\ Px = \frac{FxL - My}{Fz} & ( 1 )\end{matrix}$$

$$\begin{matrix}[ Math . 2 ] & \\ Py = \frac{FyL + Mx}{Fz} & ( 2 )\end{matrix}$$

$$\begin{matrix}[ Math . 3 ] & \\ Pz = 0 & ( 3 )\end{matrix}$$

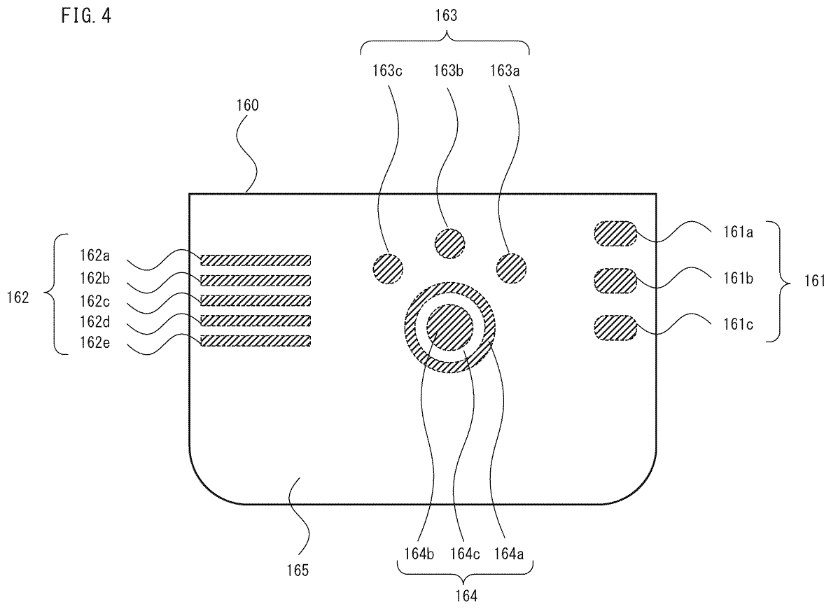

FIG. 4 is a diagram illustrating an example of the operation map 160 of the acquisition unit 130. The operation map 160 has the operation regions overlapping coordinates of the protruded portions and/or the depressed portions serving as the operation units provided on the base material 110. To each of the operation regions of the operation map 160, the predetermined function for operating the ultrasonic diagnostic apparatus 1 is allocated. In a case of FIG. 4, an operation region 161, an operation region 162, an operation region 163, and an operation region 164 respectively correspond to the slide switch unit 111, the slider unit 112, the dial unit 113, and the dial trackball unit 114. Meanwhile, a region of the planar portion of the surface of the base material 110 other than the operation units serves as a non-operation region 165 that does not receive an operation by the operator.

The acquisition unit 130 acquires the position information indicating the point of action and the operation information indicating an operation on one of the operation units on the basis of the force and/or moment from the detection sensor 122. The acquisition unit 130 has a function of outputting, on the basis of the acquired position information and operation information, an operation signal indicating the predetermined function allocated to each of the operation regions of the operation map 160 to the display control unit 60 or the system control unit 80 of the ultrasonic diagnostic apparatus 1. At the same time, the acquisition unit 130 has a function of outputting the vibration signal corresponding to the operation information of the predetermined function to the vibration control unit 140.

For example, the acquisition unit 130 has a determination function of determining that, when determining that the point of action of a finger of the operator is in the operation region 161 from the position information, the operator is touching the slide switch unit 111. The acquisition unit 130 has a function of acquiring, when the operator operates the slide switch unit 111 with his finger, a direction of movement of the force from the detection sensor 122 as the operation information and outputting the operation signal for the slide switch to the system control unit 80. The acquisition unit 130 also has a function of outputting, to the vibration control unit 140, the vibration signal corresponding to an operation of the slide switch by the operator.

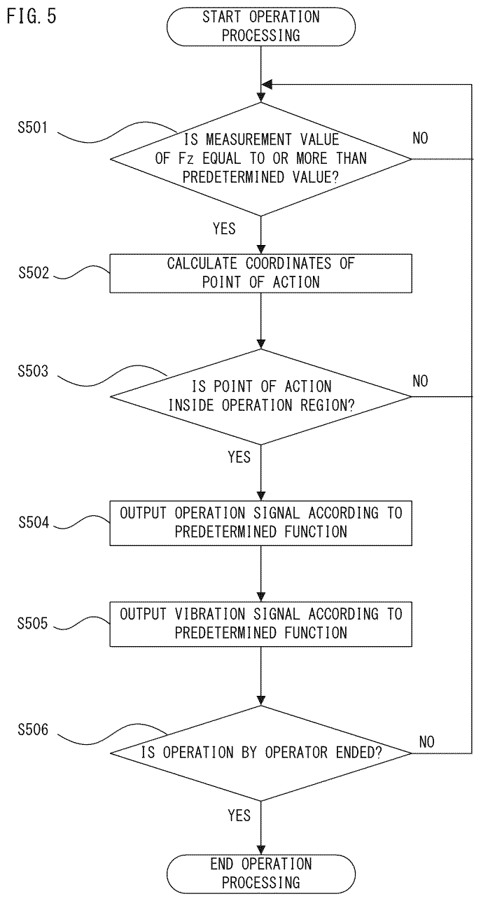

FIG. 5 is a flow chart of operation processing performed by the acquisition unit 130 in the electronic device 100. In Step S501, the acquisition unit 130 has a function of determining whether or not a measurement value of the force Fz in a Zs-axis direction from the detection sensor 122 (six-axis force sensor) is equal to or more than a predetermined value. When the measurement value is equal to or more than the predetermined value, the acquisition unit 130 advances the processing to Step S502. When the measurement value is equal to or less than the predetermined value, the acquisition unit 130 does not perform subsequent processing, and repeats the processing in Step S501. By thus providing a threshold for the determination of the measurement value by the detection sensor 122, it is possible to prevent an erroneous operation when the operator touches the base material by mistake.

In Step S502, the acquisition unit 130 uses Expressions (1) to (3) shown above to calculate the coordinates P of the point of action of the operator. Then, in Step S503, the acquisition unit 130 determines, on the basis of the calculated coordinates, whether or not the point of action of the finger of the operator is inside any of the operation regions of the operation map 160. When the point of action is outside the operation regions, the acquisition unit 130 returns the processing to Step S501. Meanwhile, when the point of action is inside any of the operation regions, the acquisition unit 130 advances the processing to Step S504.

In Step S504, the acquisition unit 130 acquires the operation information on the basis of the force and/or moment measured by the detection sensor 122, and outputs the operation signal indicating the function allocated to the operation region overlapping the point of action to the display control unit 60 or the system control unit 80. The display control unit 60 or the system control unit 80 controls execution of the predetermined function on the basis of the operation signal.

In Step S505, the acquisition unit 130 transmits the vibration signal corresponding to the predetermined function to the vibration control unit 140. The vibration control unit 140 drives, on the basis of the vibration signal, the vibration actuator serving as the vibration generation unit 150 to give the force feedback that notifies the operator that the operation is actually performed. This allows the operator to recognize that he is operating the electronic device by touching the slide switch unit 111.

In Step S506, the acquisition unit 130 determines whether or not the operation by the operator is ended. For example, when the detection by the detection unit 120 has not been performed continuously for a predetermined time, the acquisition unit 130 determines that the operation by the operator is ended, and ends the processing of the present flow chart. When the detection by the detection unit 120 has been performed within a predetermined time, the acquisition unit 130 determines that the operation is not ended, and returns the processing to Step S501.

Next, a description will be given of the predetermined functions of the ultrasonic diagnostic apparatus 1 corresponding to the individual protruded portions and depressed portions each included in the operation units of the base material 110.

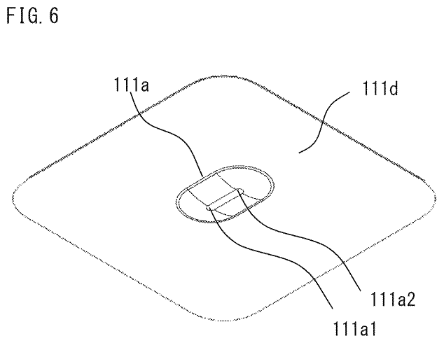

(Slide Switch) FIG. 6 is an enlarged perspective view of an ellipsoidal depressed portion serving as the operation unit provided in the base material 110, which is the depressed portion 111a illustrated in FIG. 2. The operation area of the operation map 160 in this case is an operation region 161a corresponding to the depressed portion as illustrated in FIG. 4 and, when the point of action of the finger indicated by the position information described above is inside the operation region, it is determined by the acquisition unit 130 that there is an operation. The depressed portion 111a, which is the ellipsoidal depressed portion, has a switching function, similarly to a mechanical slide switch.

As illustrated in FIG. 6, a planar portion 111d of the base material 110 around the depressed portion 111a is not an operation region of the operation map 160. Accordingly, as illustrated in FIG. 3, when the operator touches the planar portion 111d, coordinates of a touch position and a force are detected, but the acquisition unit 130 does not determine that the touch on the planar portion 111d is an operation by the operator, and does not receive the operation by the touch. In other words, the planar portion 111d of the base material 110 does not have the switching function of the depressed portion 111a.

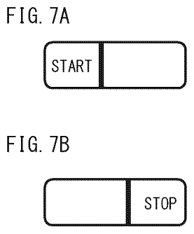

FIG. 7 illustrates an example of a slide switch on the GUI displayed by the display control unit according to the present embodiment. For example, to the depressed portion 111a serving as the slide switch unit, a function of switching between the start and stop of the transmission of the ultrasonic wave by the ultrasonic probe 11 is allocated. A state of the depressed portion 111a illustrated in FIG. 7A is a state where the start of the transmission of the ultrasonic wave by the ultrasonic probe 11 is received. A “START” position illustrated in FIG. 7A corresponds to an end 111al of the ellipsoidal depressed portion in FIG. 6.

As illustrated in FIG. 7A, when the “START” is displayed on the GUI, the operator touches the end 111a1 (FIG. 6) of the ellipsoidal depressed portion with his finger and slides the finger directly to an opposite end 111a2 (FIG. 6). At this time, the acquisition unit 130 outputs the operation signal for a switching instruction to the ultrasonic probe 11. The ultrasonic probe 11 starts to transmit the ultrasonic wave according to the operation signal input thereto from the acquisition unit 130. At this time, the display on the GUI in in FIG. 7A switches from the start to the stop, as illustrated in FIG. 7B. Note that the display position of the “STOP” in FIG. 7B corresponds to the end 111a2 of the ellipsoidal depressed portion. When the operator of the acquisition unit touches the end 111a2 of the ellipsoidal depressed portion with his finger and slides the finger directly to the opposite end 111a1 herein, the acquisition unit 130 outputs the operation signal for the switching instruction to the ultrasonic probe 11. The ultrasonic probe 11 stops the transmission of the ultrasonic wave according to the operation signal input thereto from the acquisition unit 130. Thus, the ultrasonic probe 11 switches between the start/stop of the transmission of the ultrasonic wave according to the operation signal input thereto from the acquisition unit 130. Note that the slide switch unit 111 is not limited to the ellipsoidal depressed-type stereoscopic portion, and may also be a substantially rectangular or protruded-type stereoscopic portion as long as a shape thereof is similar to that on the GUI in FIG. 7 so as to allow the operator to easily recognize the slide switch unit 111 when the operator touches the slide switch unit 111 with his finger.

When the finger of the operator touches the end 111al or 111a2 of an ellipsoid or the operator slides the finger to switch between the start/stop of the transmission of the ultrasonic wave, the vibration control unit 140 causes the vibration generation unit 150 to vibrate the receiver 121, i.e., the base material 110. More specifically, the vibration generation unit 150 vibrates a portion with the end 111al or 111a2 of the ellipsoid. As a result, the operator can receive the force feedback when operating the depressed portion 111a to switch between the start/stop of the transmission of the ultrasonic wave. This allows the operator to recognize that he is operating the electronic device. When the operator touches the planar portion 111d, under the control of the vibration control unit 140, the vibration generation unit 150 does not vibrate the receiver 121, i.e., the base material 110 (planar portion 111d). This allows the operator to recognize that he is not operating the electronic device by touching the planar portion 111d.

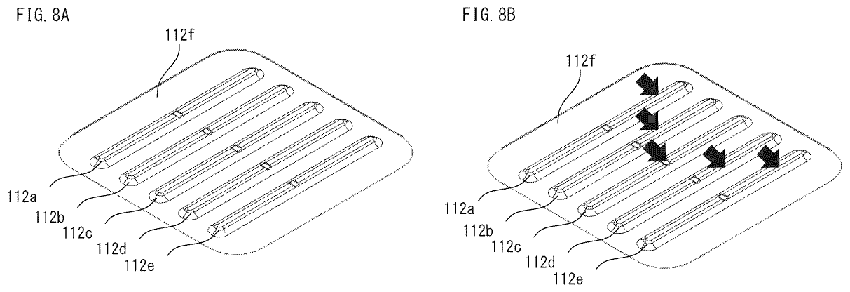

(Slider) FIGS. 8A and 8B are enlarged perspective views of the base material having a total of two or more linear protruded portions, which is the slider unit 112 in FIG. 2. As illustrated in FIG. 4, the operation region in the operation map 160 is the operation region 162 corresponding to the protruded portions 112a to 112e and, when the point of action of the finger is inside the operation region, the acquisition unit 130 determines that there is an operation on the slider unit 112. The protruded portions 112a to 112e have shapes extending in one direction in plan view of the base material, and has a slider function, similarly to a mechanical slide-type variable resistor. For example, on the operation map 160, to the slider unit 112, a function of adjusting a gain (brightness) at each depth corresponding to the ultrasonic image is allocated.

As also illustrated in FIGS. 8A and 8B, a planar portion 112f of the base material 110 around the protruded portions 112a to 112e is not an operation region of the operation map 160. Accordingly, as illustrated in FIG. 3, when the operator touches the planar portion 112f, coordinates of a touch position and a force are detected, but the acquisition unit 130 does not determine that the touch on the planar portion 112f is an operation by the operator, and does not receive the operation by the touch. In other words, the planar portion 112f of the base material 110 does not have the slider function of each of the protruded portions 112a to 112e.

When the operator touches any of the protruded portions 112a to 112e with his finger and directly slides the finger, the acquisition unit 130 outputs the operation signal for an instruction to move to a slide position in response to a finger operation. Meanwhile, when the operator does not slide the finger over the protruded portions 112a to 112e and taps a point over the protruded portions 112a to 112e, the acquisition unit 130 acquires position coordinates of the tapped point of action, and outputs the operation signal for moving a knob to the position indicated by the acquired coordinates on the GUI.

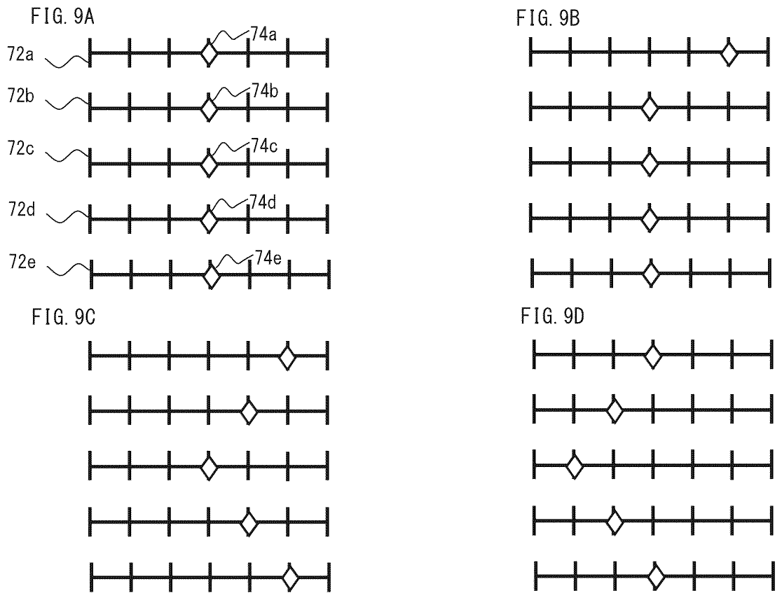

FIGS. 9A to 9D are diagrams illustrating an example of display of the sliders on the GUI of the display control unit according to the present embodiment. As illustrated in FIG. 9A, on the GUI, for the individual protruded portions 112a to 112e, slide bars 72a to 72e serving as sliders corresponding thereto and knobs 74a to 74d indicating current positions on the sliders are displayed. Note that, in FIGS. 8B to 9D, reference signs of the slide bars and the knobs are omitted. The knobs 74a to 74e are indices indicating values of variable amounts at the current positions resulting from operations performed on the slide bars 72a to 72e. For example, from the initial state in FIG. 9A, when the operator touches a center of the protruded portion 112a located at an uppermost position with his finger and performs a slide operation thereon to the right side, the knob 74a moves as in FIG. 9B. Meanwhile, when the operator performs no slide operation and sequentially taps positions indicated by individual arrows illustrated in FIG. 8B with his finger, on the GUI, the knobs 74a to 74e move to the tapped positions as in FIG. 9C. Thus, by moving the knobs 74a to 74e according to points of action, which are positions tapped by the finger, the operator can swiftly change the setting of each of the sliders.

By way of example, when the operator taps twice (double-taps) the position of the protruded portion 112a corresponding to the knob 74a after setting each of the sliders, current relative positions of the individual knobs 74a to 74e are fixed. Accordingly, when the operator slides any of the protruded portions 112a to 112e with his finger to the left side, on the GUI, all the knobs 74a to 74e simultaneously move to the left side, while maintaining the relative positions, as illustrated in FIG. 9D. Thus, the operator can easily change the setting using the slide bars 72a to 72e. Note that, when the relative positions of the individual knobs 74a to 74e are to be fixed, it may be possible to perform not only double-tapping, but also long-pressing or the like on any of the knobs 74a to 74e. Alternatively, it may also be possible to provide the same slide switch unit as the slide switch unit 111 in the vicinity of the protruded portions 112a to 112e (in this case, the “START” and “STOP” illustrated in FIGS. 7A and 7B are respectively changed to “FIX” and “UNFIX”). As a result, by operating the slide switch unit, the operator can switch the position of each of the knobs 74a to 74e between fixation and movement.

Note that the protruded portions 112a to 112e are not limited to the protruded-type stereoscopic portions, and may also be depressed-type stereoscopic portions, and may appropriately have shapes similar to those displayed on the GUI illustrated in FIGS. 9A to 9D so as to allow the operator to easily recognize the protruded portions 112a to 112e when touching the protruded portions 112a to 112e with his finger. Meanwhile, the vibration control unit 140 causes the vibration generation unit 150 to vibrate the receiver 121, i.e., the base material 110 (protruded portions 112a to 112e) when the finger touches the stereoscopic portion and when the finger slides to move the knob. As a result, the operator can receive the force feedback when operating the protruded portions 112a to 112e. This allows the operator to recognize that he is operating the electronic device. When the operator touches the planar portion 112f, under the control of the vibration control unit 140, the vibration generation unit 150 does not vibrate the receiver 121, i.e., the base material 110 (planar portion 112f). This allows the operator to recognize that he is not operating the electronic device by touching the planar portion 112f.

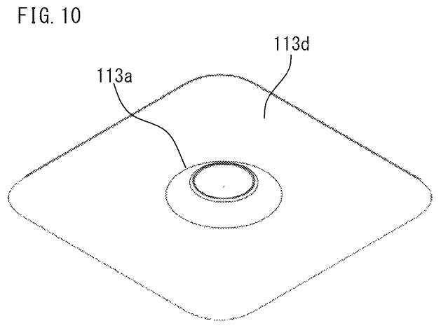

(Dial) FIG. 10 is an enlarged perspective view of the base material having the cone-shaped protruded portion 113a, which corresponds to the protruded portion 113a forming the dial unit 113 in FIG. 2. Note that each of the protruded portions 113b and 113c is configured similarly to the protruded portion 113a. As illustrated in FIG. 4, the operation region in the operation map 160 is the operation region 163 corresponding to the protruded portions 113a to 113c, and the acquisition unit 130 determines that there is an operation on the dial unit 113 when the point of action of the finger is inside the operation region. Each of the protruded portions 113a to 113c has a function of switching a switch by performing a rotating operation in the same manner as in a mechanical dial switch. For example, in the operation map 160, to the dial unit 113, a function of changing a gain (brightness) of the amplifier of the transmission/reception unit 10 is allocated.

As also illustrated in FIG. 10, a planar portion 113d of the base material 110 around the protruded portion 113a is not an operation region of the operation map 160. Accordingly, as illustrated in FIG. 3, when the operator touches the planar portion 113d, coordinates of a touch position and a force are detected, but the acquisition unit 130 does not determine that the touch on the planar portion 113d is an operation by the operator, and does not receive the operation by the touch. In other words, the planar portion 113d of the base material 110 does not have a function of switching the switch of the protruded portion 113a. Note that respective planar portions around the protruded portions 113b and 113c are also configured as regions which do not receive an operation, similarly to the planar portion 113d described above.

When the operator pinches a cone-shaped side surface of any of the protruded portions 113a to 113c with his fingers and attempts to rotate the protruded portion by twisting the protruded portion, the detection sensor 122 detects a moment Mz around the Zs-axis at the corresponding one of the protruded portions 113a to 113c. Then, the acquisition unit 130 outputs a signal for a switch switching instruction on the basis of the detected moment, and the gain is changed. Meanwhile, when the operator pinches any of the protruded portions 113a to 113c with his fingers or twists any of the protruded portions 113a to 113c to change the gain, the vibration control unit 140 causes the vibration generation unit 150 to vibrate the receiver 121, i.e., the base material 110 (any of the protruded portions 113a to 113c). Thus, the operator can receive the force feedback when operating any of the protruded portions 113a to 113c. This allows the operator to recognize that he is operating the electronic device. In addition, when the operator touches the planar portion 113d, under the control of the vibration control unit 140, the vibration generation unit 150 does not vibrate the receiver 121, i.e., the base material 110 (planar portion 113d). This allows the operator to recognize that he is not operating the electronic device by touching the planar portion 113d.

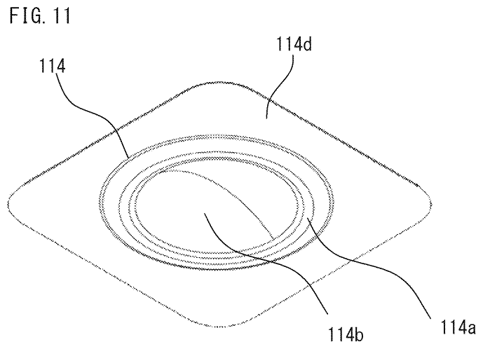

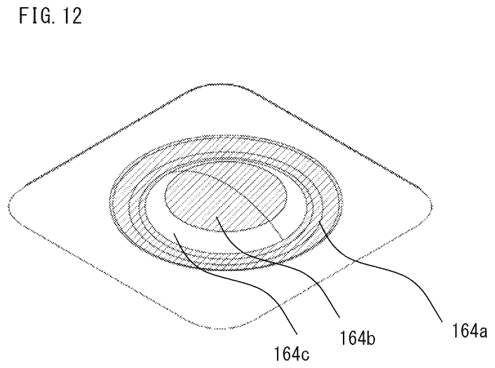

(Dial Trackball) FIG. 11 is an enlarged perspective view of the base material having the operation unit obtained by combining the circular depressed portion 114a and the protruded portion 114b hemispherically protruded inside the depressed portion 114a, which corresponds to the dial trackball unit 114 in FIG. 2. FIG. 12 is a perspective view illustrating an operation region 164a, an operation region 164b, and a non-operation region 164c each corresponding to the dial trackball unit 114 in the operation map 160.

As illustrated in FIGS. 11 and 12, the protruded portion 114b corresponding to the operation region 164b has a circular shape formed of a portion of a hemisphere. Meanwhile, the non-operation region 164c provided so as to surround the operation region 164b is an annular region provided in an inclined surface of the protruded portion 114b. In addition, the depressed portion 114a corresponding to the operation region 164a provided so as to surround the non-operation region 164c is also an annular region.

As illustrated in FIG. 12, in the operation map 160, the operation region of the dial trackball unit 114 is configured to include the two operation regions 164a and 164b and the non-operation region 164c which is provided between the operation regions 164a and 164b and does not receive an operation. When the operator operates the dial trackball unit 114 with his finger, in a case where the point of action of the finger is inside any of the operation regions 164a and 164b, the acquisition unit 130 determines that there is an operation on the dial trackball unit 114.

As also illustrated in FIG. 11, a planar portion 114d of the base material 110 around the dial trackball unit 114 is not an operation region of the operation map 160. Accordingly, as illustrated in FIG. 3, when the operator touches the planar portion 114d, coordinates of a touch position and a force are detected, but the acquisition unit 130 does not determine that the touch on the planar portion 114d is an operation by the operator, and does not receive the operation by the touch. In other words, the planar portion 114d of the base material 110 does not have the function of the dial trackball unit which is shared between the depressed portion 114a and a protruded portion 114b.

As will be described later, a function allocated to the operation region 164b of the protruded portion 114b and a function allocated to the operation region 164a of the depressed portion 114a are different from each other. Accordingly, the operator selectively uses the protruded portion 114b and the depressed portion 114a when operating the dial trackball unit 114 to be able to switchably perform the different functions. It may also be possible to paint the respective portions of the protruded portion 114b and the depressed portion 114a corresponding to the operations regions 164a and 164b in different colors or perform different surface treatment (treatment providing different surface roughnesses by embossing, smoothing, or the like). Thus, during the operation of the dial trackball unit 114, it is possible to prevent a visual and/or tactile erroneous operation. Note that the hemispherical shape of the protruded portion 114b is not limited to a shape obtained by halving a sphere in parallel with the planar portion as illustrated in FIGS. 11 and 12, and a shape obtained by, e.g., cutting a portion of the sphere with any plane may also be used.

In addition, the depressed portion 114a has a function of performing the operation in a rotating direction in the same manner as in the mechanical dial switch to switch the switch. For example, to the operation region 164a of the depressed portion 114a, a function of changing a size of a display image is allocated. Meanwhile, the protruded portion 114b has a function of a pointing device, a function of scrolling a screen, a function of rotating display, and the like in the same manner as in a mechanical trackball. For example, to the operation region 164b of the protruded portion 114b, such functions as movement of a cursor, determination of a position of the ROI, placement of a measurement tool, and rotation of a three-dimensional stereoscopic image are allocated.

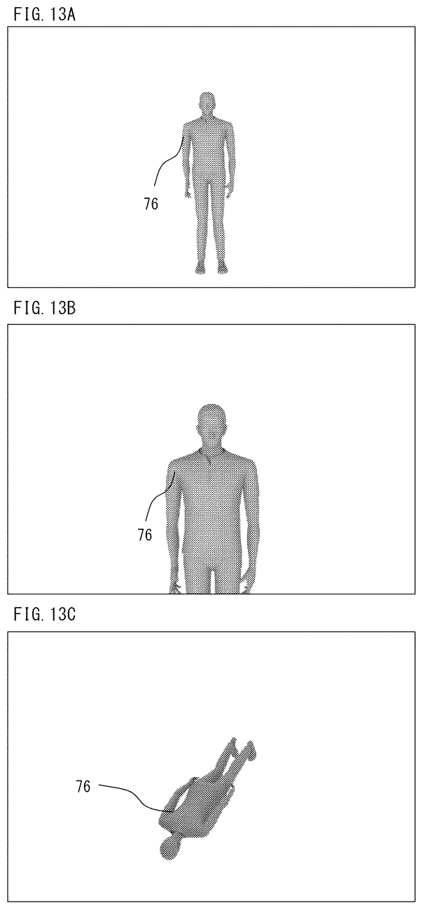

Next, referring to FIGS. 13A to 13C, a description will be given of a three-dimensional stereoscopic image displayed on the display unit 70 when each of the operation units described above is operated. In the example illustrated in FIGS. 13A to 13C, a model 76 is displayed on the display unit 70. A display screen of a three-dimensional stereoscopic image illustrated by way of example in FIGS. 13A to 13C is an example of an operation screen for operating the electronic device.

When the operator touches the depressed portion 114a with his finger and directly moves the finger in a circumferential direction of the depressed portion 114a, the acquisition unit 130 outputs the operation signal according to a travel distance of the finger. For example, a case is assumed in which, in the operation map 160, a function of enlarging/reducing a three-dimensional stereoscopic image is allocated to the operation region 164a of the depressed portion 114a. In this case, when the operator moves the finger that has touched the depressed portion 114a in a clockwise direction, the model 76 displayed on the display unit 70 is enlarged as in FIGS. 13A to 13B. Conversely, when the operator moves the finger that has touched the depressed portion 114a in a counterclockwise direction, the model 76 displayed on the display unit 70 is reduced.

Meanwhile, when the operator touches the protruded portion 114b with his finger and directly moves the finger over the protruded portion 114b, the acquisition unit 130 outputs the operation signal according to the travel distance of the finger. For example, a case is assumed in which, in the operation map 160, the function of rotating the three-dimensional stereoscopic image is applied to the operation region 164b of the protruded portion 114b. In this case, when the operator downwardly moves the finger that has touched the protruded portion 114b from over the protruded portion 114b in FIG. 2B, as illustrated in FIGS. 13A to 13C, the model 76 displayed on the display unit 70 is rotated such that a head region moves to the front, while leg regions move to the back. Thus, the display control unit 60 controls the display of the operation screen by the display unit 70 so as to display a result of an operation indicated by operation information.

The vibration control unit 140 vibrates the vibration generation unit 150 when the operator touches the depressed portion 114a with his finger or when the operator moves the finger that has touched the depressed portion 114a over a predetermined distance in the circumferential direction. In addition, the vibration control unit 140 causes the vibration generation unit 150 to vibrate the receiver 121, i.e., the base material 110 (the depressed portion 114a and the protruded portion 114b) when the operator touches the protruded portion 114b with his finger or the operator moves the finger that has touched the protruded portion 114b over a predetermined distance over a hemisphere. Meanwhile, when the finger of the operator touches a region corresponding to the non-operation region 164c located between the depressed portion 114a and the protruded portion 114b, the vibration control unit 140 does not cause the vibration generation unit 150 to vibrate the receiver 121, i.e., the base material 110. More specifically, the vibration control unit 140 does not cause the vibration generation unit 150 to vibrate a portion with the planar portion 114d between the depressed portion 114a and the protruded portion 114b. In other words, the non-operation region 164c functions as a region where the control unit does not receive an operation by the operator between regions of a surface of the base material to which different functions of at least one of the protruded portion and the depressed portion are set. This allows the operator to recognize the movement of the finger from the operation region (in a state where the vibration generation unit 150 causes vibration) to the non-operation region (in a state where the vibration generation unit 150 does not cause vibration) and can prevent the finger from touching another operation region and performing an erroneous operation.

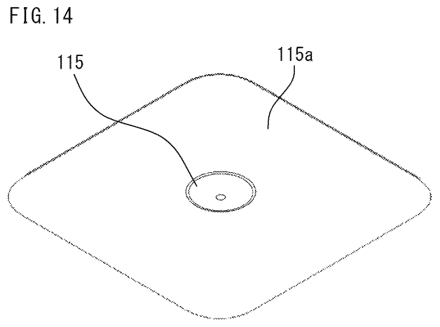

Next, referring to FIGS. 14 to 17, a description will be given of a modification of the protruded portion and the depressed portion each provided on the base material described above.

(Push Switch) FIG. 14 is a perspective view of a push switch unit 115, which is a hemispherical depressed portion. In the operation map 160, an operation region of the push switch unit 115 corresponds to a region formed with the depressed portion. The push switch unit 115 has a function of pushing a portion having a depressed shape and thereby allowing ON/OFF switching, similarly to a mechanical push switch. When the operator pushes the push switch unit 115 with his finger, the acquisition unit 130 outputs an ON/OFF signal. Note that the push switch unit 115 is not limited to the depressed portion, and may also be formed of another shape such as that of a protruded portion. The vibration control unit 140 causes the vibration generation unit 150 to vibrate the receiver 121, i.e., the base material 110 (the push switch unit 115) when the operator pushes the depressed portion of the push switch unit 115 with his finger. As a result, the operator can receive the force feedback when operating the push switch unit 115. This allows the operator to recognize that he is operating the electronic device.

As illustrated in FIG. 14, the planar portion 115a of the base material 110 around the push switch unit 115 is not an operation region of the operation map 160. Accordingly, as illustrated in FIG. 3, when the operator touches the planar portion 115a, coordinates of a touch position and a force are detected, but the acquisition unit 130 does not determine that the touch on the planar portion 115a is an operation by the operator, and does not receive the operation by the touch. In other words, the planar portion 115a of the base material 110 does not have the function of the push switch unit 115. In addition, when the operator touches the planar portion 115a, under the control of the vibration control unit 140, the vibration generation unit 150 does not vibrate the receiver 121, i.e., the base material 110 (planar portion 115a). This allows the operator to recognize that he is not operating the electronic device by touching the planar portion 115a.

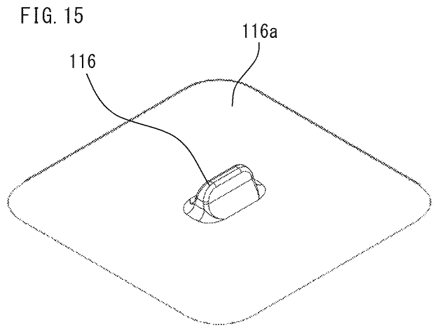

(Knob Dial) FIG. 15 is a perspective view of a knob dial unit 116, which is a rectangular protruded portion. In the operation map 160, an operation region of the knob dial unit 116 corresponds to a region where the protruded portion is formed. The knob dial unit 116 has a function of switching a switch by performing a rotating operation on a portion having a protruded shape in the same manner as in a mechanical knob dial switch. When the operator pinches the protruded portion with his fingers and rotates the protruded portion by twisting the protruded portion, the detection sensor 122 detects the moment Mz around the Zs-axis at the protruded portion, and the acquisition unit 130 outputs a signal for the switch switching instruction. Meanwhile, when the operator pinches the protruded portion of the knob dial unit 116 with his fingers or twists the protruded portion, the vibration control unit 140 causes the vibration generation unit 150 to vibrate the receiver 121, i.e., the base material 110 (dial unit 116). As a result, when operating the dial unit 116, the operator can receive the force feedback. This allows the operator to recognize that he is operating the electronic device.

As illustrated in FIG. 15, a planar portion 116a of the base material 110 around the dial unit 116 is not an operation region of the operation map 160. Accordingly, as illustrated in FIG. 3, when the operator touches the planar portion 116a, coordinates of a touch position and a force are detected, but the acquisition unit 130 does not determine that the touch on the planar portion 116a is an operation by the operator, and does not receive the operation by the touch. In other words, the planar portion 116a of the base material 110 does not have the function of the dial unit 116. In addition, when the operator touches the planar portion 116a, under the control of the vibration control unit 140, the vibration generation unit 150 does not vibrate the receiver 121, i.e., the base material 110 (planar portion 116a). This allows the operator to recognize that he is not operating the electronic device by touching the planar portion 116a.

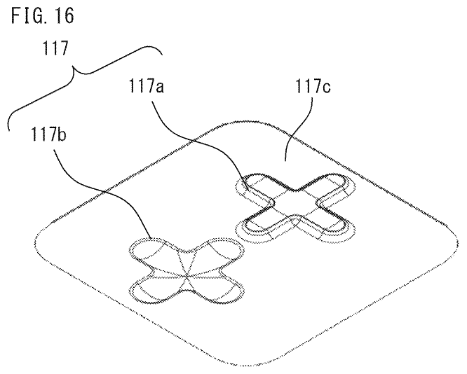

(Cross Key) FIG. 16 is a perspective view of a cross key unit 117 including a pair of a protruded portion 117a and a depressed portion 117b each having a cross key shape. In the operation map 160, an operation region of the cross key unit 117 corresponds to a region where the protruded portion 117a is formed and a region where the depressed portion 117b is formed. Each of the protruded portion 117a and the depressed portion 117b of the cross key unit 117 has a function of pushing a leading end thereof in any of four directions and thereby indicating the direction in the same manner as in a mechanical cross key switch. When the operator pushes the protruded portion 117a or the depressed portion 117b, the acquisition unit 130 outputs an instruction signal in the direction of the cross key corresponding to the pushed position. Note that the protruded portion 117a and the depressed portion 117b have different stereoscopic shapes, but have the same function of the cross key switch. For example, when there are two types of functions as operations to be performed on a target displayed on the display unit 70, two cross keys having different shapes, such as a combination of the protruded portion 117a and the depressed portion 117b in FIG. 16, are arranged, and the different functions are assigned to the respective cross keys in the operation map 160. Accordingly, by touching the cross key unit 117 with his finger without visually recognizing the cross key unit 117, the operator can recognize which one of the functions of the protruded portion 117a and the depressed portion 117b the operated cross key has, and can prevent an erroneous operation. Meanwhile, when the operator has pushed any of end portions in the four directions of the protruded portion 117a or the depressed portion 117b with his finger, the vibration control unit 140 causes the vibration generation unit 150 to vibrate the receiver 121, i.e., the base material 110 (the protruded portion 117a or the depressed portion 117b). As a result, the operator can receive the force feedback when operating the cross key unit 117. This allows the operator to recognize that he is operating the electronic device.

As illustrated in FIG. 16, a planar portion 117c of the base material 110 around the cross key unit 117 is not an operation region of the operation map 160. Accordingly, as illustrated in FIG. 3, when the operator touches the planar portion 117c, coordinates of a touch position and a force are detected, but the acquisition unit 130 does not determine that the touch on the planar portion 117c is an operation by the operator, and does not receive the operation by the touch. In other words, the planar portion 117c of the base material 110 does not have the function of the cross key unit 117. In addition, when the operator touches the planar portion 117c, under the control of the vibration control unit 140, the vibration generation unit 150 does not vibrate the receiver 121, i.e., the base material 110 (planar portion 117c). This allows the operator to recognize that he is not operating the electronic device by touching the planar portion 117c.

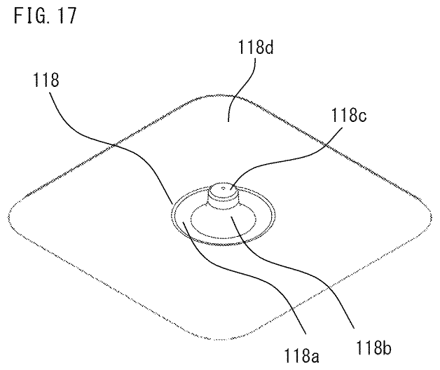

(Dial Pointing) FIG. 17 is a perspective view of the dial pointing unit 118 including a depressed portion 118a and a protruded portion 118b having an elongated cylindrical shape inside the depressed portion 118a. In the operation map 160, an operation region of the dial pointing unit 118 corresponds to a region where the protruded portion 118b is formed. Functions that can be performed by operating the protruded portion 118b of the dial pointing unit 118 have two modes. A first mode is a function of a dial in the same manner as in the dial unit 113, while a second mode is a function of a pointing device. In addition, by way of example, by double-tapping an upper surface 118c of the protruded portion 118b, the operator can switch between the first mode and the second mode. When the operator operates the dial pointing unit 118, under the control of the vibration control unit 140, the vibration generation unit 150 vibrates the receiver 121, i.e., the base material 110 (the dial pointing unit 118). As a result, when operating the dial pointing unit 118, the operator can receive the force feedback. This allows the operator to recognize that he is operating the electronic device.

As illustrated in FIG. 17, a planar portion 118d of the base material 110 around the dial pointing unit 118 is not an operation region of the operation map 160. Accordingly, as illustrated in FIG. 3, when the operator touches the planar portion 118d, coordinates of a touch position and a force are detected, but the acquisition unit 130 does not determine that the touch on the planar portion 118d is an operation by the operator, and does not receive the operation by the touch. In other words, the planar portion 118d of the base material 110 does not have the dial function of the dial pointing unit 118. When the operator touches the planar portion 118d, under the control of the vibration control unit 140, the vibration generation unit 150 does not vibrate the receiver 121, i.e., the base material 110 (planar portion 118d). This allows the operator to recognize that he is not operating the electronic device by touching the planar portion 118d.

In the first mode, in the same manner as in the dial unit 113, when the operator moves the protruded portion 118b, the detection sensor 122 detects the moment Mz around the Zs-axis of the protruded portion 118b, and the acquisition unit 130 outputs the signal for the switch switching instruction. In the second mode, when the operator pushes the protruded portion 118b, the detection sensor 122 detects a direction in which the protruded portion 118b is pushed, and the acquisition unit 130 outputs the operation signal according to the direction.

Additionally, in the first mode, when the operator pinches the protruded portion 118b or twists the protruded portion 118b, the vibration control unit 140 causes the vibration generation unit 150 to vibrate. Meanwhile, in the second mode, the vibration control unit 140 does not cause the vibration generation unit 150 to vibrate even when the protruded portion 118b is operated in the same manner in which a typical pointing device does not give a force feedback.

As described heretofore, the electronic device 100 of the present embodiment includes the operation units each including the protruded portion, the depressed portion, and the like on the base material 110, and thus allows the operator to identify each operation unit with his finger. Accordingly, the operator can easily recognize which operation unit he is operating without visually recognizing the various operation units on the base material 110 and improve the operability of the electronic device 100. In addition, the acquisition unit 130 determines at least one of the detected force and moment on the basis of a criterion differing from one function performed by the operation unit to another, and acquires the operation information of the operation unit by the operator.

Meanwhile, in the operation map 160, the planar portion of the surface of the base material 110 other than the regions where the operation units 111 to 114 are formed serves as the non-operation region. In other words, over the base material, between the regions to which different functions of at least one of the protruded portion and the depressed portion are set, the region where the control unit does not receive an operation by the operator is provided. More specifically, on the base material, at least two of the protruded portion and the depressed portion are formed, different functions are set to the at least two of the protruded portion and the depressed portion, and the planar portion is formed between regions with the at least two of the protruded portion and the depressed portion. As a result, even when the operator unintentionally slides his finger from the operation units 111 to 114 to touch the planar portion of the surface of the base material 110, the electronic device 100 does not receive an operation. Thus, in the electronic device 100, a risk of erroneous selection of the operation unit or an erroneous operation can be avoided. In addition, as the respective shapes of the operation units 111 to 114 formed on the base material 110, simple shapes are used. Therefore, it is possible to easily clean the operation units 111 to 114 with cloth or the like and keep the base material 110 clean.

Note that the shapes described above, functions according thereto, and a vibration method resulting in the force feedback are examples, and are not limited thereto.

### Second Embodiment

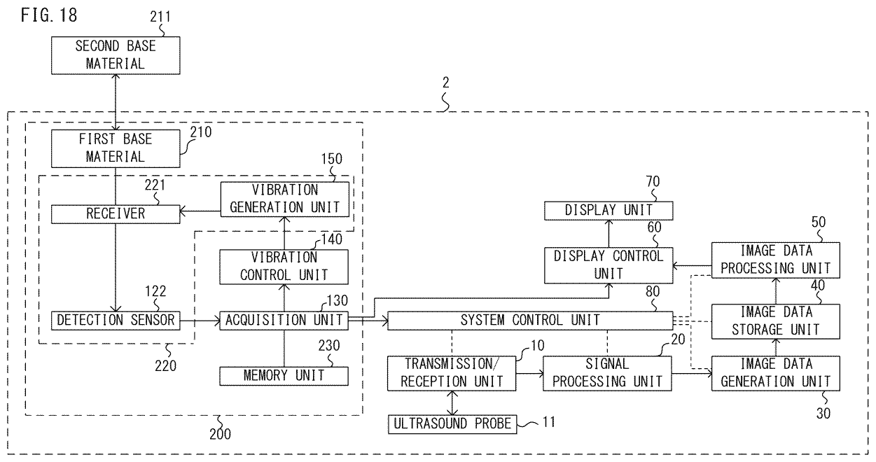

Next, referring to FIG. 18, a description will be given of an ultrasonic diagnostic apparatus having an electronic device according to the second embodiment. Note that, in the following description, the same configurations as those in the first embodiment are denoted by the same reference signs, and a detailed description thereof is omitted.

FIG. 18 is a block diagram illustrating a configuration of an ultrasonic diagnostic apparatus 2 according to the second embodiment. As also illustrated in FIG. 18, an electronic device 200 includes a first base material 210, a detection unit 220, the acquisition unit 130, the vibration control unit 140, the vibration generation unit 150, and a memory unit 230.

As will be described later, the first base material 210 is attached to a receiver 221 of the detection unit 220 to be replaceable with a second base material 211. In the same manner as on the base material 110 in FIG. 2, on the first base material 210, the slide switch unit 111, the slider unit 112, the dial unit 113, and the dial trackball unit 114 are disposed. Accordingly, an operation map corresponding to the first base material 210 is the same as the operation map 160 in FIG. 4, and a description thereof is omitted. Meanwhile, on the second base material 211, operation units having the same functions as those of the operation units disposed on the first base material 210 are disposed at positions different from those on the first base material 210. Furthermore, on the second base material 211, uneven shapes different from those of the operation units of the first base material 210, e.g., the push switch unit 115 are disposed and, on the first base material 210 and the second base material 211, the uneven shapes of the operation units and the positions of the operation units are different. Note that each of the base materials 210 and 211 may be made appropriately of a material that is not easily deformed even when the operator applies a force thereto, and it may also be possible to form the base materials 210 and 211 by performing molding using a 3D printer or surface treatment such as embossing depending on a size of a hand of the operator, a dominant hand, and ease of use.

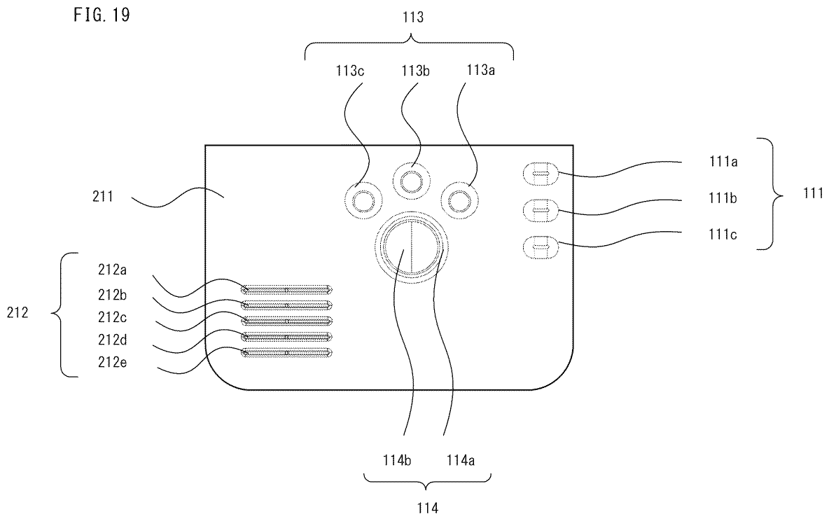

FIG. 19 is a top view illustrating an example of the various operation units of the second base material 211. On the second base material 211, a position of a slider unit 212 is different from that on the first base material 210 illustrated in FIG. 2B. Accordingly, on the second base material, at least one of the protruded portion and the depressed portion each having the same function as that of the at least one of the protruded portion and the depressed portion each disposed on the first base material is disposed at a position different from that on the first base material. Additionally, on the second base material 211, the slide switch unit 111, the dial unit 113, and the dial trackball unit 114 are disposed at the same positions as those of the individual operation units on the first base material 210. Note that a function of each of the operation units of the second base material 211 is the same as that of each of the operation units of the first base material 210, and therefore a detailed description of the function is omitted herein.

In the present embodiment, in the memory unit 230, operation maps corresponding to the respective base materials 210 and 211 are stored. As will be described later, when the base material is detached and another base material is newly attached, the acquisition unit 130 acquires, from the memory unit 230, the operation map corresponding to the newly attached base material. Thus, the acquisition unit 130 also has a function of a control unit that specifies function information corresponding to the base material on the basis of a characteristic of the base material detected by the detection unit. For example, when the first base material is detected by the detection unit, the acquisition unit 130 specifies the function information corresponding to the first base material and, when the second base material is detected by the detection unit, the acquisition unit 130 specifies the function information corresponding to the second base material.

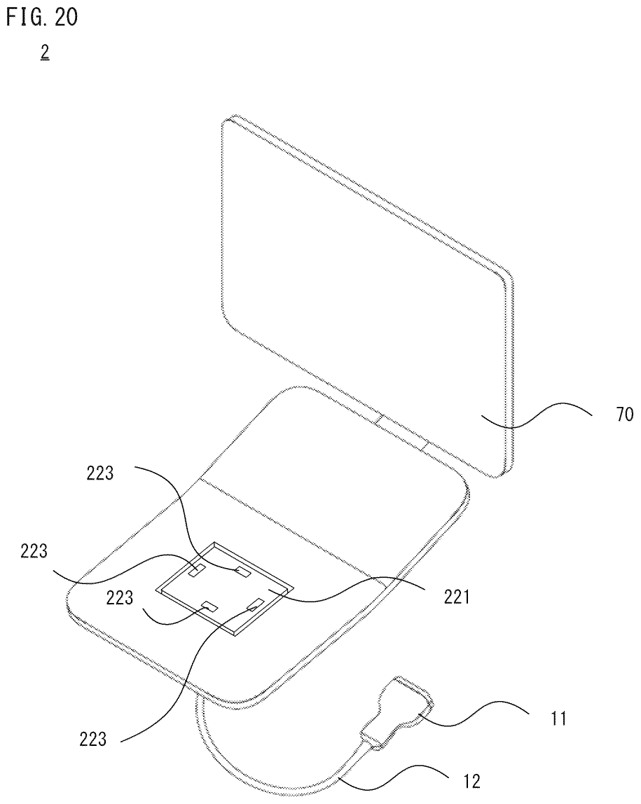

FIG. 20 is a perspective view of the ultrasonic diagnostic apparatus 2 in a state where the first base material 210 is detached. The first base material 210 is removably attached to the receiver 221 of the detection unit 220. As illustrated in FIG. 20, when the first base material 210 is detached, on the detection sensor 122 not shown, which is the six-axis force sensor, the receiver 221 appears to be visually recognizable. On the receiver 221, a magnet 223 for attaching the base materials 210 and 211 is disposed. In addition, on a back surface of each of the first base material 210 and the second base material 211 also, a magnet or a metal thin plate is disposed at a position to be attached to the receiver 221. As a result, even when the base material is contaminated, the base material can easily be replaced, which allows each of the operation units of the base material to be kept clean. In addition, it is also possible to replace the base materials on which the operation units are disposed at different positions or different surface treatments are performed on the operation units depending on the size of the hand of the operator, the dominant hand, and ease of use. Alternatively, it is also possible to replace the base material with a base material to which an appropriate operation map is applied depending on a use situation. Thus, in the electronic device 200 in the present embodiment, by merely replacing the base material without replacing the electronic device and consequently replacing the ultrasonic diagnostic apparatus, it is possible to improve the operability of the operator and reduce time by promptly changing a diagnosis method.

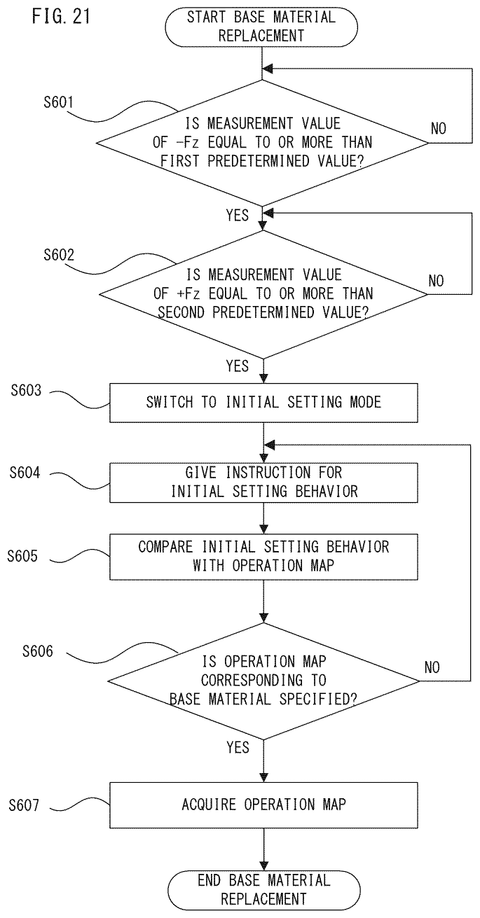

FIG. 21 is a flow chart of processing in which the acquisition unit 130 of the electronic device 200 makes initial settings when base material replacement is performed. In Step S601, the acquisition unit 130 determines whether or not a measurement value of −Fz serving as a pulling force in the Zs-axis direction from the six-axis force sensor, which is the detection sensor 122, is equal to or more than a first predetermined value. In a case where the first base material 210 is attached to the receiver 221, when the operator is to detach the first base material 210 from the receiver 221, a force to pull off an adsorption force of the magnet 223 is required. Accordingly, the detection sensor 122 detects −Fz serving as the pulling force in the Zs-axis direction. Therefore, the first predetermined value may be set appropriately to be substantially equal to the adsorption force of the magnet. The acquisition unit 130 advances the processing to Step S602 when the measurement value from the detection sensor 122 is equal to or more than the first predetermined value.

In Step S602, the acquisition unit 130 determines whether or not a measurement value of +Fz serving as a pushing force in the Zs-axis direction from the detection sensor 122 is equal to or more than a second predetermined value. When another base material other than the first base material 210, e.g., the second base material 211 is attached to the receiver 221, the second base material 211 is attracted by an adsorption force of the magnet 223 to the receiver 221 and attached thereto. At this time, the detection sensor 122, which is the six-axis force sensor, detects a positive pressure and outputs the pressure as the measurement value. The acquisition unit 130 advances the processing to Step S603 when the measurement value from the detection sensor 122 is equal to or more than the second predetermined value.

In Step S603, the acquisition unit 130 recognizes that base material replacement was performed, and switches the behavior of the electronic device 200 to an initial setting mode for the operation map. In the initial setting mode, a state is established where the acquisition unit 130 has not acquired the operation map.

Then, in Step S604, the acquisition unit 130 transmits a signal for switching the display on the display unit 70 to the initial setting mode to the display control unit 60. The display control unit 60 performs display to prompt the operator to perform an initial setting behavior on the display unit 70. In a specific example, the display control unit 60 performs display to require the operator to touch any of the operation units of the second base material 211 on the display unit 70. Then, in Step S605, the acquisition unit 130 compares the initial setting behavior performed by the operator in Step S604 with each of the operation maps stored in the memory unit 230 to specify the operation map corresponding to the base material attached to the receiver 221. Thus, the acquisition unit 130 specifies the function information corresponding to the base material on the basis of an operation by the operator. Then, the acquisition unit 130 performs initial setting by using the specified operation map.

Specifically, in Step S604, the operator touches the second base material 211 attached to the receiver 221 with his finger to perform the initial setting behavior. Then, in Step S605, the acquisition unit 130 acquires the point of action of the finger or the direction of the force on the basis of the initial setting behavior of the operator in Step S604, and compares the point of action of the finger and the direction of the force with the operation map of each of the base materials stored in the memory unit 230. The initial setting behavior of the operator mentioned herein is a behavior of touching any of the operation units of the base material with his finger. For example, the operator attaches the second base material 211 and performs a behavior (the initial setting behavior) of sliding the finger from an end of a slider unit 212a to another end thereof in FIG. 19. Then, the acquisition unit 130 collates the operation map on the basis of determination of whether or not the point of action of the finger overlaps the operation region of each of the operation maps stored in the memory unit.

In Step S606, the acquisition unit 130 determines whether or not the initial setting behavior of the operator matches the operation map of any of the base materials stored in the memory unit as a result of collating the operation map. For example, when the operator has performed a behavior of sliding his finger from one end of the slider unit 212a to another end thereof, the acquisition unit 130 determines that the operation region of the operation map corresponding to the second base material 211 overlaps the point of action of the finger, and recognizes that the second base material 211 has been attached to the receiver 221. Meanwhile, when the operation map having the operation region overlapping the point of action of the finger is not stored in the memory unit, the acquisition unit 130 returns the processing to Step S604, controls the display control unit 60, and performs display to require the operator to perform the initial setting behavior again on the display unit 70. Note that when the same protruded portion or the same depressed portion is disposed at the same position on any of the different base materials and the different base material matches another operation map, it is appropriate to prevent the position of the operation unit to be used for the initial setting behavior from overlapping the position of the operation unit on the other base material.

In Step S607, the acquisition unit 130 acquires the operation map matching the base material attached to the receiver 221 from the memory unit 230. Then, the acquisition unit 130 ends the initial setting mode, and ends the processing of the present flow chart.

As described above, by storing the operation maps corresponding to the plurality of respective base materials in the memory unit, an appropriate operation map is applied to the base material attached at the replacement of the base material. As a result, it is possible to replace the base material with a base material having an operation unit arrangement and shapes, surface treatment, and the like which are appropriate for each operator and use an operation map appropriate for the post-replacement base material. Therefore, with the electronic device according to the present embodiment, it is possible to improve the operability of each of the operation units of the base material by the operator. In addition, even when the base material is contaminated, the base material can easily be replaced, which allows the base material to be kept clean. Even when, e.g., software to be applied to the ultrasonic diagnostic apparatus is updated and an operation method is changed, as long as a new base material and a new operation map are prepared, it is possible to appropriately operate even the apparatus for which the operation method has been changed without replacing the entire apparatus. In other words, by replacing the base material of the electronic device, it is possible to change the design of the ultrasonic diagnostic apparatus at low cost.

### Third Embodiment

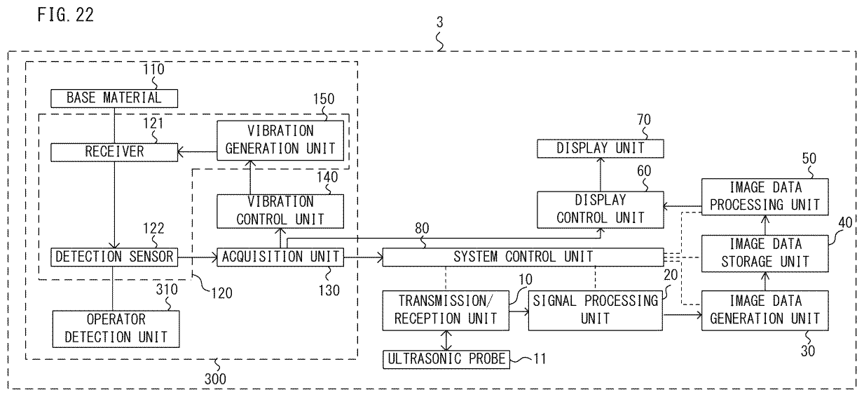

Next, referring to FIG. 22, a description will be given of an ultrasonic diagnostic apparatus having an electronic device according to the third embodiment. Note that, in the following description, the same configurations as those in the first embodiment are denoted by the same reference signs, and a detailed description thereof is omitted. FIG. 22 is a block diagram illustrating a configuration of an ultrasonic diagnostic apparatus 3 according to the present embodiment. As illustrated in FIG. 22, an electronic device 300 includes the base material 110, the detection unit 120, the acquisition unit 130, the vibration control unit 140, the vibration generation unit 150, and an operator detection unit 310.

The operator detection unit 310 is formed of a sensor that detects contact with or proximity to the operator, which is a pressure sensor serving as a contact detection unit provided separately from the detection unit 120. The operator detection unit 310 is disposed at a position on the base material which is proximate to at least one of a protruded portion and a depressed portion. When detecting the contact with or proximity to the operator, the operator detection unit 310 transmits a signal for starting operation detection to the detection sensor 122 of the detection unit 120. Then, the detection sensor 122 starts to detect a force acting on the base material 110.

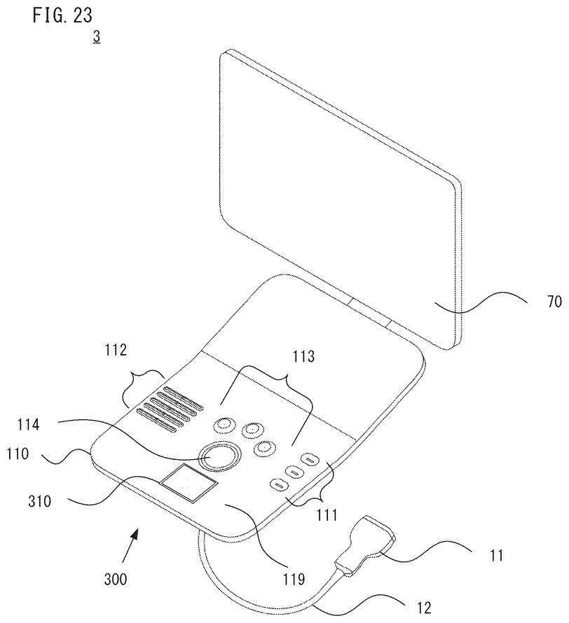

FIG. 23 is a perspective view of the ultrasonic diagnostic apparatus 3 configured to include the electronic device 300 including the operator detection unit 310. The base material 110 of the ultrasonic diagnostic apparatus 3 has the operator detection unit 310 provided under the dial trackball unit 114, and the operator detection unit 310 functions also as a palm rest. The operator detection unit 310 is provided with, e.g., a pressure sensor not shown and, when the operator places his wrist on the operator detection unit 310 so as to operate the base material 110, the pressure sensor of the operator detection unit 310 detects a pressure. When detecting the pressure, the operator detection unit 310 transmits the signal for starting operation detection to the detection sensor 122, and the detection sensor 122 detects a force acting on the base material. Meanwhile, when the pressure sensor detects no pressure, the detection sensor 122 does not detect a force acting on the base material 110, and therefore the electronic device 300 does not receive an operation by the operator. Thus, when the contact with or proximity to the operator is detected by the contact detection unit, the acquisition unit 130 serves as the control unit and validates an operation on at least one of the protruded portion and the depressed portion of the base material by the operator.

For example, when the operator places his wrist on the operator detection unit 310 and operates the slide switch unit 111, the operator detection unit 310 detects the contact with or proximity to the operator, and accordingly the electronic device 300 receives an operation by the operator. A slide function of the slide switch unit 111 is validated and, e.g., the transmission of the ultrasonic wave can be started/stopped. In addition, when the operator places his wrist on the operator detection unit 310 and operates the slide switch unit 111, under the control of the vibration control unit 140, the vibration generation unit 150 vibrates the receiver 121, i.e., the base material 110 (the slide switch unit 111). As a result, when operating the slide switch unit 111, the operator can receive the force feedback. This allows the operator to recognize that he is operating the electronic device.

Meanwhile, when, e.g., the operator does not place his wrist on the operator detection unit 310 and operates the slide switch unit 111, the operator detection unit 310 does not detect the contact with or proximity to the operator, and accordingly the electronic device 300 does not receive an operation by the operator. The slide function of the slide switch unit 111 is invalidated and, e.g., the transmission of the ultrasonic wave cannot be started/stopped. When the operator does not place his wrist on the operator detection unit 310 and operates the slide switch unit 111, under the control of the vibration control unit 140, the vibration generation unit 150 does not vibrate the receiver 121, i.e., the base material 110 (the slide switch unit 111). This allows the operator to recognize that he is not operating the electronic device.

Likewise, when, e.g., the operator places his wrist on the operator detection unit 310 and operates the slider unit 112, the operator detection unit 310 detects the contact with or proximity to the operator, and accordingly the electronic device 300 receives an operation by the operator. An adjustment function of the slider unit 112 is validated and, e.g., gain adjustment of an ultrasonic image can be performed. In addition, when the operator places his wrist on the operator detection unit 310 and operates the slider unit 112, under the control of the vibration control unit 140, the vibration generation unit 150 vibrates the receiver 121, i.e., the base material 110 (the slider unit 112). As a result, when operating the slider unit 112, the operator can receive the force feedback. This allows the operator to recognize that he is operating the electronic device.

Meanwhile, when, e.g., the operator does not place his wrist on the operator detection unit 310 and operates the slider unit 112, the operator detection unit 310 does not detect the contact with or proximity to the operator, and accordingly the electronic device 300 does not receive an operation by the operator. The adjustment function of the slider unit 112 is invalidated and, e.g., the gain adjustment of the ultrasonic image cannot be performed. When the operator does not place his wrist on the operator detection unit 310 and operates the slider unit 112, under the control of the vibration control unit 140, the vibration generation unit 150 does not vibrate the receiver 121, i.e., the base material 110 (the slider unit 112). This allows the operator to recognize that he is not operating the electronic device.

Likewise, when, e.g., the operator places his wrist on the operator detection unit 310 and operates the dial unit 113, the operator detection unit 310 detects the contact with or proximity to the operator, and accordingly the electronic device 300 receives an operation by the operator. A changing function of the dial unit 113 is validated and, e.g., a gain of the amplifier of the transmission/reception unit 10 can be changed. In addition, when the operator places his wrist on the operator detection unit 310 and operates the dial unit 113, under the control of the vibration control unit 140, the vibration generation unit 150 vibrates the receiver 121, i.e., the base material 110 (the dial unit 113). As a result, when operating the dial unit 113, the operator can receive the force feedback. This allows the operator to recognize that he is operating the electronic device.

Meanwhile, when, e.g., the operator does not place his wrist on the operator detection unit 310 and operates the dial unit 113, the operator detection unit 310 does not detect the contact with or proximity to the operator, and accordingly the electronic device 300 does not receive an operation by the operator. The changing function of the dial unit 113 is invalidated and, e.g., the gain of the amplifier of the transmission/reception unit 10 cannot be changed. When the operator does not place his wrist on the operator detection unit 310 and operates the dial unit 113, under the control of the vibration control unit 140, the vibration generation unit 150 does not vibrate the receiver 121, i.e., the base material 110 (the dial unit 113). This allows the operator to recognize that he is not operating the electronic device.

Likewise, when, e.g., the operator places his wrist on the operator detection unit 310 and operates the dial trackball unit 114, the operator detection unit 310 detects the contact with or proximity to the operator, and accordingly the electronic device 300 receives an operation by the operator. A changing function of the dial trackball unit 114 is validated and, e.g., a size of a display image can be changed. In addition, when the operator places his wrist on the operator detection unit 310 and operates the dial trackball unit 114, under the control of the vibration control unit 140, the vibration generation unit 150 vibrates the receiver 121, i.e., the base material 110 (the dial trackball unit 114). As a result, when operating the dial trackball unit 114, the operator can receive the force feedback. This allows the operator to recognize that he is operating the electronic device.

Meanwhile, when, e.g., the operator does not place his wrist on the operator detection unit 310 and operates the dial trackball unit 114, the operator detection unit 310 does not detect the contact with or proximity to the operator, and accordingly the electronic device 300 does not receive an operation by the operator. The changing function of the dial trackball unit 114 is invalidated and, e.g., the size of the display image cannot be changed. When the operator does not place his wrist on the operator detection unit 310 and operates the dial trackball unit 114, under the control of the vibration control unit 140, the vibration generation unit 150 does not vibrate the receiver 121, i.e., the base material 110 (the dial trackball unit 114). This allows the operator to recognize that he is not operating the electronic device.

Likewise, when, e.g., the operator places his wrist on the operator detection unit 310 and operates the push switch unit 115, the operator detection unit 310 detects the contact with or proximity to the operator, and accordingly the electronic device 300 receives an operation by the operator. A switching function of the push switch unit 115 is validated, and it is possible to, e.g., switchably turn ON/OFF a function to be performed by the electronic device 300. In addition, when the operator places his wrist on the operator detection unit 310 and operates the push switch unit 115, under the control of the vibration control unit 140, the vibration generation unit 150 vibrates the receiver 121, i.e., the base material 110 (the push switch unit 115). As a result, when operating the push switch unit 115, the operator can receive the force feedback. This allows the operator to recognize that he is operating the electronic device.

Meanwhile, when, e.g., the operator does not place his wrist on the operator detection unit 310 and operates the push switch unit 115, the operator detection unit 310 does not detect the contact with or proximity to the operator, and accordingly the electronic device 300 does not receive an operation by the operator. The switching function of the push switch unit 115 is invalidated, and it is impossible to, e.g., switchably turn ON/OFF the function to be performed by the electronic device 300. When the operator does not place his wrist on the operator detection unit 310 and operates the push switch unit 115, under the control of the vibration control unit 140, the vibration generation unit 150 does not vibrate the receiver 121, i.e., the base material 110 (the push switch unit 115). This allows the operator to recognize that he is not operating the electronic device.

Likewise, when, e.g., the operator places his wrist on the operator detection unit 310 and operates the knob dial unit 116, the operator detection unit 310 detects the contact with or proximity to the operator, and accordingly the electronic device 300 receives an operation by the operator. A switching function of the knob dial unit 116 is validated, and it is possible to, e.g., switch a switch in the electronic device 300. In addition, when the operator places his wrist on the operator detection unit 310 and operates the knob dial unit 116, under the control of the vibration control unit 140, the vibration generation unit 150 vibrates the receiver 121, i.e., the base material 110 (the knob dial unit 116). As a result, when operating the knob dial unit 116, the operator can receive the force feedback. This allows the operator to recognize that he is operating the electronic device.

Meanwhile, when, e.g., the operator does not place his wrist on the operator detection unit 310 and operates the knob dial unit 116, the operator detection unit 310 does not detect the contact with or proximity to the operator, and accordingly the electronic device 300 does not receive an operation by the operator. The switching function of the knob dial unit 116 is invalidated, and it is impossible to, e.g., switch the switch in the electronic device 300. When the operator does not place his wrist on the operator detection unit 310 and operates the knob dial unit 116, under the control of the vibration control unit 140, the vibration generation unit 150 does not vibrate the receiver 121, i.e., the base material 110 (the knob dial unit 116). This allows the operator to recognize that he is not operating the electronic device.

Likewise, when, e.g., the operator places his wrist on the operator detection unit 310 and operates the cross key unit 117, the operator detection unit 310 detects the contact with or proximity to the operator, and accordingly the electronic device 300 receives an operation by the operator. A function of the cross key switch of the cross key unit 117 is validated, and it is possible to, e.g., operate a target displayed on the display unit 70. In addition, when the operator places his wrist on the operator detection unit 310 and operates the cross key unit 117, under the control of the vibration control unit 140, the vibration generation unit 150 vibrates the receiver 121, i.e., the base material 110 (the cross key unit 117). As a result, when operating the cross key unit 117, the operator can receive the force feedback. This allows the operator to recognize that he is operating the electronic device.

Meanwhile, when, e.g., the operator does not place his wrist on the operator detection unit 310 and operates the cross key unit 117, the operator detection unit 310 does not detect the contact with or proximity to the operator, and accordingly the electronic device 300 does not receive an operation by the operator. The function of the cross key switch of the cross key unit 117 is invalidated, and it is impossible to, e.g., operate the target displayed on the display unit 70. When the operator does not place his wrist on the operator detection unit 310 and operates the cross key unit 117, under the control of the vibration control unit 140, the vibration generation unit 150 does not vibrate the receiver 121, i.e., the base material 110 (the cross key unit 117). This allows the operator to recognize that he is not operating the electronic device.

Likewise, when, e.g., the operator places his wrist on the operator detection unit 310 and operates the dial pointing unit 118, the operator detection unit 310 detects the contact with or proximity to the operator, and accordingly the electronic device 300 receives an operation by the operator. A dial function and a pointing device function of the dial pointing unit 118 are validated, and it is possible to, e.g., switch a switch in the electronic device 300. In addition, when the operator places his wrist on the operator detection unit 310 and operates the dial pointing unit 118, under the control of the vibration control unit 140, the vibration generation unit 150 vibrates the receiver 121, i.e., the base material 110 (the dial pointing unit 118). As a result, when operating the dial pointing unit 118, the operator can receive the force feedback. This allows the operator to recognize that he is operating the electronic device.

Meanwhile, when, e.g., the operator does not place his wrist on the operator detection unit 310 and operates the dial pointing unit 118, the operator detection unit 310 does not detect the contact with or proximity to the operator, and accordingly the electronic device 300 does not receive an operation by the operator. The dial function and the pointing device function of the dial pointing unit 118 are invalidated, and it is impossible to, e.g., switch the switch in the electronic device 300. When the operator does not place his wrist on the operator detection unit 310 and operates the dial pointing unit 118, under the control of the vibration control unit 140, the vibration generation unit 150 does not vibrate the receiver 121, i.e., the base material 110 (the dial pointing unit 118). This allows the operator to recognize that he is not operating the electronic device.

Thus, the operator detection unit 310 functions as a contact detection unit accompanying an operation performed on at least one of the protruded portion and the depressed portion by the operator to detect the contact with or proximity to the operator. As a result, even when the operator unintentionally touches the operation unit of the base material 110, unless the operator detection unit 310 has detected the contact with or proximity to the operator, an operation performed on the operation unit is not received, and therefore it is possible to prevent an erroneous operation by the operator and allow a reliable operation. Note that the operator detection unit 310 is not limited to the detection of a pressure of a hand of the operator performing an operation using the pressure sensor, and may also be a non-contact human detecting sensor such as an infrared sensor that detects the presence of the hand of the operator performing the operation with an infrared ray or a touch sensor that detects the presence of the hand of the operator with static electricity.

When the operator places his wrist on the operator detection unit 310 and operates the operation unit of the base material 110, under the control of the vibration control unit 140, the vibration generation unit 150 vibrates the receiver 121, i.e., the base material 110. As a result, the operator can receive the force feedback when operating the operation unit of the base material 110. This allows the operator to recognize that he is operating the electronic device. When the operator does not place his wrist on the operator detection unit 310 and operates the operation unit of the base material 110 or when the operator touches the planar portion 119, under the control of the vibration control unit 140, the vibration generation unit 150 does not vibrate the receiver 121, i.e., the base material 110 (planar portion 119). This allows the operator to recognize that he is not operating the electronic device.

According to the present disclosure, it is possible to enhance an operability of each of an electronic device, a medical instrument, and an ultrasonic diagnostic apparatus which are operated by an operator.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An electronic device comprising:
a base material;
a processor; and
a memory storing a program which, when executed by the processor, causes the image processing apparatus to:
execute detection processing of detecting an operation performed on the base material by an operator;
execute acquisition processing of acquiring, on the basis of an output from the detection processing according to the detection of the operation, position information related to the operation on the base material; and
execute control processing of controlling a behavior of the electronic device on the basis of the position information acquired by the acquisition processing, wherein
the base material is provided with a region where in the control processing, the operation by the operator is not received.

2. The electronic device according to claim 1, wherein the base material is formed with at least one of a protruded portion and a depressed portion.

3. The electronic device according to claim 1, wherein, between regions of a surface of the base material to which different functions are set, the region where in the control processing, the operation by the operator is not received is provided.

4. The electronic device according to claim 1, wherein in the control processing, the behavior of the electronic device is controlled on the basis of the position information and function information related to a function to be executed by the operation performed on the base material.

5. The electronic device according to claim 2, wherein the region where in the control processing, the operation by the operator is not received is provided at a position corresponding to at least one of the protruded portion and the depressed portion.

6. The electronic device according to claim 1, wherein in the control processing, it is determined whether or not a position indicated by the acquired position information overlaps the region where the operation by the operator is not received, and, on the basis of a result of the determination, it is determined whether or not the operation by the operator is to be received.

7. The electronic device according to claim 1, wherein in the detection processing at least one of a force and a moment each exerted on the base material by the operator is detected.

8. The electronic device according to claim 7, wherein
in the acquisition processing, operation information on the operation performed on the base material by the operator is acquired on the basis of at least one of the force and the moment each detected by the detection processing, and
in the control processing, the behavior of the electronic device is controlled on the basis of the operation information acquired by the acquisition processing.

9. The electronic device according to claim 8, wherein
the program further causes the image processing apparatus to:
execute vibration generation processing of giving a vibration feedback to the operator; and
execute vibration control processing of controlling the vibration generation processing on the basis of the operation information acquired by the acquisition processing.

10. The electronic device according to claim 1, wherein
the program further causes the image processing apparatus to:
execute contact detection processing of detecting contact with or proximity to the operator resulting from the operation performed on the base material by the operator.

11. The electronic device according to claim 2, wherein a region of a surface of the base material other than a region thereof where at least one of the protruded portion and the depressed portion is formed is provided with the region where in the control processing the operation by the operator is not received.

12. The electronic device according to claim 11, wherein a planar portion of the surface of the base material other than the region thereof where at least one of the protruded portion and the depressed portion is formed is the region where in the control processing the operation by the operator is not received.

13. The electronic device according to claim 12, wherein
at least two of the protruded portion and the depressed portion are formed on the surface of the base material, and different functions are set to the at least two of the protruded portion and the depressed portion, and
the planar portion is formed between at least two regions of the protruded portion and the depressed portion.

14. A medical instrument comprising:
a base material;
a processor; and
a memory storing a program which, when executed by the processor, causes the medical instrument to:
execute detection processing of detecting an operation performed on the base material by an operator;
execute acquisition processing of acquiring, on the basis of an output from the detection processing according to the detection of the operation, position information related to the operation on the base material; and
execute control processing of controlling a behavior of the medical instrument on the basis of the position information acquired by the acquisition processing, wherein
the base material is provided with a region where in the control processing, the operation by the operator is not received.

15. An ultrasonic diagnostic apparatus comprising:
a base material;
a processor; and
a memory storing a program which, when executed by the processor, causes the ultrasonic diagnostic apparatus to:
execute detection processing of detecting an operation performed on the base material by an operator;
execute acquisition processing of acquiring, on the basis of an output from the detection processing according to the detection of the operation, position information related to the operation on the base material; and
execute control processing of controlling a behavior of the ultrasonic diagnostic apparatus on the basis of the position information acquired by the acquisition processing, wherein
the base material is provided with a region where in the control processing, the operation by the operator is not received.

16. The ultrasonic diagnostic apparatus according to claim 15, wherein the behavior of the ultrasonic diagnostic apparatus includes at least transmission and reception of an ultrasonic wave by the ultrasonic diagnostic apparatus, gain adjustment in an ultrasonic image, changing of a size of the ultrasonic image, and rotation of the ultrasonic image.

17. The electronic device according to claim 1, wherein the base material is replaceably attached to the electronic device.

18. The electronic device according to claim 17, wherein in the control processing, the behavior of the electronic device is controlled on the basis of the position information and function information related to a function to be executed by the operation performed on the base material.

19. The electronic device according to claim 18, wherein in the control processing, the function information for each base material attached to the electronic device is switched, and the electronic device is controlled on the basis of the function information after the switching.

20. The electronic device according to claim 18, wherein in the control processing, the function information is specified corresponding to the base material.

21. The electronic device according to claim 20, wherein in the control processing, the function information is specified corresponding to a first base material when the first base material is detected by the detection processing, and specifies the function information corresponding to a second base material when the second base material different from the first base material is detected by the detection processing.

22. The medical instrument according to claim 14, wherein the base material is replaceably attached to the medical instrument.

23. The ultrasonic diagnostic apparatus according to claim 15, wherein the base material is replaceably attached to the ultrasonic diagnostic apparatus.
